SEQUENCE LISTING (13 sequences)

SEQ ID NO: 1 - RNA, 22 nt, Artificial Sequence
  microRNA-122-5p mature mimic (miR-122)
uggagugugacaaugguguuug

SEQ ID NO: 2 - RNA, 22 nt, Artificial Sequence
  MUTATED miR-122
aggagugugacaaugguguuug

SEQ ID NO: 3 - RNA, 22 nt, Artificial Sequence
  MUTATED miR-122
ucgagugugacaaugguguuug

SEQ ID NO: 4 - RNA, 22 nt, Artificial Sequence
  MUTATED miR-122
uccagugugacaaugguguuug

SEQ ID NO: 5 - RNA, 22 nt, Artificial Sequence
  MUTATED miR-122
ucgugugugacaaugguguuug

SEQ ID NO: 6 - RNA, 22 nt, Artificial Sequence
  MUTATED miR-122
ucgaaugugacaaugguguuug

SEQ ID NO: 7 - RNA, 22 nt, Artificial Sequence
  MUTATED miR-122
ucgaguguguugaugguguuug

SEQ ID NO: 8 - RNA, 22 nt, Artificial Sequence
  MUTATED miR-122
ucgagugugacaauggugaaag

SEQ ID NO: 9 - RNA, 22 nt, Artificial Sequence
  MUTATED miR-122
ucgagugugacagcgguguuug

SEQ ID NO: 10 - RNA, 22 nt, Artificial Sequence
  microRNA-451-5p mature mimic sequence (miR-451)
aaaccguuaccauuacugaguu

SEQ ID NO: 11 - DNA, 16 bp, Artificial Sequence
  miR-451 INHIBITOR
agtaagggtaacggtt

SEQ ID NO: 12 - DNA, 15 bp, Artificial Sequence
  miR-451 INHIBITOR
gtaatggtaacggtt

SEQ ID NO: 13 - RNA, 22 nt, Artificial Sequence
  miR-451 INHIBITOR
ucaacaucagucugauaagcua

(19) United States
(12) Patent Application Publication
(10) Pub. No.: US 2023/0407304 A1
(43) Pub. Date: Dec. 21, 2023

(54) USE OF MICRORNA INHIBITION TO PREVENT AND TREAT OSTEOARTHRITIS AND OTHER INFLAMMATORY DISEASES

(71) Applicant: VIRGINIA COMMONWEALTH UNIVERSITY, Richmond, VA (US)

(72) Inventors: Zvi SCHWARTZ, HENRICO, VA (US); Barbara D. BOYAN, HENRICO, VA (US); Kayla M. SCOTT, RICHMOND, VA (US); Joshua D. COHEN, RICHMOND, VA (US)

(21) Appl. No.: 18/253,529

(22) PCT Filed: Nov. 19, 2021
(86) PCT No.: PCT/US2021/060054

§ 371 Date: May 18, 2023

Related U.S. Application Data
(60) Provisional application No. 63/225,932, filed on Jul. 26, 2021.
(60) Provisional application No. 63/216,199, filed on Jun. 29, 2021.
(60) Provisional application No. 63/158,004, filed on Mar. 8, 2021.
(60) Provisional application No. 63/155,396, filed on Mar. 2, 2021.
(60) Provisional application No. 63/115,767, filed on Nov. 19, 2020.

(51) Int. Cl. C12N 15/113; A61K 31/7105; A61P 19/02

(52) U.S. Cl. CPC C12N 15/113; A61K 31/7105; A61P 19/02

(57) ABSTRACT

Inflammatory processes, particularly those arising from trauma or injury to the region of a bone joint, are causative of osteoarthritis (OA). A composition comprising microRNA-122, microRNA-122 mimic, and/or an inhibitor of microRNA-451 reduces or inhibits inflammatory mediators in articular chondrocytes and related cells. When administered to a subject at risk of developing OA, development of OA and/or progression of OA disease are inhibited, and cartilaginous structures are preserved in the joint. Since joint injury is associated with subsequent development of OA, the treatment is particularly suitable following an acute injury to a joint.

### CROSS-REFERENCE TO RELATED APPLICATIONS

This application claims the benefit of U.S. Provisional Application No. 63/115,767, filed Nov. 20, 2020; U.S. Provisional Application No. 63/155,396, filed Mar. 2, 2021; U.S. Provisional Application No. 63/158,004, filed Mar. 8, 2021; U.S. Provisional Application No. 63/216,199, filed Jun. 29, 2021; and U.S. Provisional Application No. 63/225,932, filed Jul. 26, 2021; which are hereby incorporated herein by reference in its entirety.

### FIELD OF THE INVENTION

The invention is generally related to pharmaceutical compositions and methods for inhibiting or treating osteoarthritis. The pharmaceutical compositions comprise microRNA species, mimics and/or inhibitors that reduce the inflammatory processes that contribute to the development of osteoarthritis. Administration of the pharmaceutical is particularly useful as a treatment following trauma to a joint to inhibit initiation and/or progression of osteoarthritis disease.

### BACKGROUND OF THE INVENTION

Osteoarthritis (OA) is a debilitating disease that affects over 32.5 million adults in the United States alone. Many risk factors contribute to the development of OA including age, gender, weight, genetics, joint alignment, and joint injuries. Joint injuries are of particular concern as compounding evidence indicates that 80% of anterior ligament injured knees will develop radiographic evidence of OA 5 to 15 years following the initial injury. Currently, there is no cure for OA, and physicians focus on managing symptoms until a total joint replacement (knee, shoulder, hip and others) is necessary. Compounding evidence suggests that the development of inflammation plays a key role in advancing the progression of OA Inflammatory cytokines create a positive feedback loop driving the chronic production of interleukin (IL)-1 (IL-1), tumor necrosis factor-alpha (TNF-α), IL-17, IL-18, prostaglandin E₂ (PGE2), reactive oxygen species nitric oxide (NO), as well as the synthesis of collagenases such as matrix metalloproteinase-1 (MMP-1), MMP-8, MMP-13, and aggrecanases ADAMTS4 and ADAMTS5. Together these molecules drive matrix degradation and exacerbate OA progression. Both NO and PGE2 have been directly implicated in apoptosis. Not surprisingly, OA cartilage exhibits a higher proportion of apoptotic chondrocytes as compared to normal tissue.

Specifically, IL-1β, TNF-α, and WNT/β-catenin (WNT/β-cat) signaling are dysregulated in OA. Research has indicated they drive OA progression. The elevation of IL-1β and TNF-α induce the production of MMPs, PGEs, other cytokines, and inhibit the production of ECM components (proteoglycans, collagen type II). Inhibition of TNF-α decreases OA severity in both spontaneously and surgically induced OA models in mice and in experimentally induced OA in rabbits. Additionally, TNF-α inhibition promotes the repair of osteochondral lesions and has chondroprotective effects in vivo. Interestingly, Zwerina et al. found that articular cartilage changes caused by overexpression of TNF-α are not fully blocked by either TNF-α or IL-1 inhibition alone; however, the combined blockade of both TNF-α and IL-1 lead to almost complete remission of the disease (Arthritis Rheum. 2004; 50(1):277-90). This lends credence to the established idioms of multiple aberrant pathways influencing OA progression.

MicroRNAs (miRs) have been found to operate as an intricate and multilayered regulatory system governing transcriptional control mechanisms and overarching metabolic homeostasis (see Bhaskaran et al. Vet Pathol. 2014; 51(4):759-74; also see Rottiers et al. Nat Rev Mol Cell Biol. 2012 March; 13(4):239-50). These short (19-24 nucleotides) evolutionarily conserved RNAs function as post-transcriptional regulators of protein production. Up to 60% of all human protein-coding genes are predicted to be subject to miR regulation. miRs regulate gene expression by binding to the 3′ UTR of a target mRNA which prevents translation through two mechanisms: 1) a complete binding to mRNA resulting in the destruction of the target mRNA, or 2) a partial binding to complementary mRNA inhibiting translation into protein. However, recent evidence suggests that miRNAs destabilize mRNA through deadenylation and subsequent turnover in P-bodies. Two distinct classes govern miRNA biogenesis: miRs created from overlapping introns of protein transcripts as well as those encoded in exons. As many as 50% of miRs are co-transcribed from the introns of coding genes while the rest are expressed from non-protein-coding transcripts.

miRs have been implicated in numerous disease pathologies, including cardiovascular diseases, cancer, and rheumatoid and osteoarthritis (see Miyaki et al. Genes Dev. 2010; 24(11):1173-85; Dai et al. Arthritis Res Ther. 2012 December; 14(6):R268; and Chang et al. J Cell Biochem. 2018; (September 2017):4775-82). With microRNA's being co-transcribed along with mRNAs, it is not a surprise that diseases that result in phenotypical changes have vastly different microRNA profiles from their healthy tissue counterparts. miR communication is vast; they can signal in an autocrine, paracrine, and or endocrine manner. This is due to the numerous ways miRs can be transported—in plasma by binding to high-density lipoprotein (HDL) or low-density lipoprotein (LDL), ribonucleoprotein complexes, as the cargo in matrix vesicles (MVs), or in extracellular vesicles (EVs) (see Vickers et al. Nat Cell Biol. 2011; 13(4):423-33; Qing et al. Neurorehabil Neural Repair. 2018; 32(9):765-76; Rogers & Aikawa. Arterioscler Thromb Vasc Biol. 2019; 39(12):2448-50; and Boon & Vickers. Arterioscler Thromb Vasc Biol. 2013; 33(2):186-92). Interestingly, EV cargo (miRs, DNA, proteins, and lipids) is altered in disease states to promote and drive disease progression. This is more than likely due to the phenotypical changes in the disease profile producing altered microRNA profiles.

The miR identified as miR-122 has gained attention for its role in hepatic lipid and cholesterol metabolism, hepatocyte differentiation, and liver homeostasis where it makes up 70% of all miRs expressed in the liver and has been coined a ‘liver specific’ miRNA, although we now know this its expression is not liver specific (see Liu et al. Cardiovasc Toxicol. 2020; 20(5):463-73; and Scott et al. Osteoarthr Cartil. 2020; 29:113-23). miR-122 expression has been found in liver disease and in liver fibrosis; genetic deletion of miR-122 decreased lipid metabolism, steatohepatitis, fibrosis, inflammation, and hepatocellular cancer. The specificity of miR-122 in the liver was confirmed when restoration of miR-122 in miR-122-depleted KO mice reversed liver inflammation. These data speak to the anti-inflammatory and anti-fibrotic functions of miR-122, which likely extends to other tissues. For example, HCV's replication dependence on miR-122 expression status has led to the development of Miravirsen, an inhibitor to miR-122, and it is currently being evaluated in Phase II clinical trials to treat chronic hepatitis C in patients who have not responded to the traditional treatment.

The literature has conflicting results on the role of miR-451 in the body. Low levels of miR-451 have been correlated with increased prostate cancer, gastric cancer, renal cell carcinoma, non-small cell lung cancer, breast cancer, and HCC, while others have shown over-expression of miR-451 is linked with cancer metastasis. This may be due to its ability to inhibit proliferation in numerous cancers. Transfection of miR-451 in glomerular mesangial cells decreases IL-1β, IL-6, IL-8, cell viability, and nuclear factor kappa-light-chain enhancer of activated B cells (NF-κB) signaling (see Wei et al. Biosci Rep. 2019 October; 39(10):1-11). This has been corroborated by Sun and Zhang who showed that miR-451 overexpression was able to relieve chronic inflammatory pain by inhibiting IL-1β, IL-6, and TNF-α in primary spinal microglial cells (see Cell Tissue Res. 2018; 374(3):487-95). miR-451 is a key regulator for erythroid differentiation and maturation. It does this by targeting gata2, UBE2h, and ARPP-19 (see Khordadmehr et al. J Cell Physiol. 2019; 234(12):21716-31). miR-451 knock-out mice experienced increased oxidative stress. Additionally, miR-451 upregulation has been found in autoimmune disorders such as lupus erythematosus, Graves' disease, and Hashimoto's thyroiditis. Interestingly, miR-451 is upregulated in T cells of peripheral blood circulation with rheumatoid arthritis (RA), but downregulated in RA neutrophils (see Khordadmer, supra; Smiegielska et al. Genes Immun. 2014; 15(2):115-25; and Murata et al. Arthritis Rheumatol. 2014; 66(3):549-59), speaking to the different functions of miR expression in different tissues. Previous studies that examined the packaging of miR-451 in the matrix vesicles in the growth plate did not explore its role in cartilage homeostasis. How miR-451 interfaces and communicates in the growth plate or in other cartilage is still largely unknown.

Thus, the role of miRs in OA remains unclear. There is still a need for a therapeutic that can prevent the associated increase in inflammatory modulators, decrease chondrocyte death, and encourage matrix synthesis to halt the progression of OA.

### SUMMARY

The present disclosure describes compositions and methods of treating the complex and multifaceted disease of OA. Compositions of the invention intervene in the catabolic biochemical signaling axis caused by over-modulation of inflammatory molecules that cause OA. The compositions and methods for their use promote chondrocyte proliferation and reduce the effects of IL-1β in vitro and are effective therapeutic agents in vivo for the treatment of OA.

One embodiment of the invention is a pharmaceutical composition for use as an inhibitor of osteoarthritis comprising a microRNA-122 (miR-122), an miR-122 mimic, an inhibitor of a microRNA-451 (miR-451) or any combination of these agents. The pharmaceutical composition can be administered to a subject to inhibit an inflammatory response, and more particularly to inhibit an inflammatory response in articular chondrocytes of the cartilage of a joint. Delivery of the pharmaceutical composition to a joint inhibits development or progression of osteoarthritis in the joint.

An aspect of the disclosure provides a method of treating or inhibiting osteoarthritis in a subject in need thereof, comprising the step of administering a therapeutically sufficient amount of the pharmaceutical composition comprising an miR-122, an miR-122 mimic, an inhibitor of microRNA-451 (miR-451) or any combination of these agents. The therapeutically sufficient amount of the pharmaceutical composition is able to inhibit an inflammatory response in articular chondrocytes. In yet another embodiment, the invention is a pharmaceutical composition used to inhibit OA disease progression.

In one embodiment, the subject has received an acute injury to at least one joint, and administration of the pharmaceutical composition inhibits formation of OA and progression of OA disease. The pharmaceutical composition may be administered by injection near or directly into a joint suspected of or known to be affected by OA. A treatment regimen for administration of the pharmaceutical composition may be a single prophylactic dose, particularly following an acute injury to at least one joint, or administration may be sustained for days or weeks at suitable intervals. Suitable intervals may be dependent upon the extent of an injury and may be determined by a knowledgeable practitioner to be multiple times daily, twice daily, once daily, and repeated for many days or weeks. In one embodiment, the pharmaceutical composition is administered at least twice a week for at least six weeks.

In one embodiment, the pharmaceutical composition comprises an inhibitor of microRNA-451 that modulates the miR-451-mediated inflammatory processes of osteoarthritis disease progression. The inflammatory processes may comprise a response to IL-1β and TNF-α stimulation in the articular chondrocytes and may further involve other drivers of OA progression and inflammatory cytokines, including TNF-α, NO, IL-8, MMP-13BMP-2, TGF-β, and PGE₂.

In another embodiment, the pharmaceutical composition comprises miR-122 and/or an miR-122 mimic and is administered to an affected joint where it inhibits the inflammatory response in articular chondrocytes. The miR-122 and/or mimic may be packaged with antisense-oligonucleotides, lipid nanoparticles or other means of delivery to chondrocytes of joint suspected of being affected or known to be affected by the risk of OA.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

### BRIEF DESCRIPTION OF THE DRAWINGS

The patent or application file contains at least one drawing executed in color. Copies of this patent or patent application publication with color drawings will be provided by the US Patent and Trademark Office upon request and payment of the necessary fee.

The accompanying drawings, which are incorporated in and constitute a part of this specification, illustrate embodiments of the invention and, together with a general description of the invention given above, and the detailed description given below, serve to explain the invention.

FIG. 1 shows a diagram of cytokines involved in the molecular pathogenesis of OA that occurs due to mechanical forces to a joint.

FIGS. 2A and 2B show experimental design of two animal models of OA. FIG. 2A shows an experimental design to produce a model of unilateral ACLT in 9-10-week-old Sprague Dawley rats that were treated with 24R,25. Left legs served as contralateral controls (Contralateral Con). Right legs received weekly injections of a vehicle or 24R,25. Two cohorts were examined: 1) a 5-week treatment group that received injections for the first 5 weeks following ACLT surgery and did not receive treatments for the remaining 5 weeks, and 2) a 10-week treatment group that received injections for the entire duration of the study. An ACLT group that did not receive treatments served as a positive control. Animals were killed on day 70 and hind-limbs were collected for micro-CT and histology. FIG. 2B shows an experimental design to produce a model of bilateral ACLT in 9-10-week-old Sprague Dawley rats that were treated with 24R,25. Left legs received weekly injections of the vehicle and right legs received weekly injections of 24R,25. Serum was collected on day 0 before surgery and on day 70. Following serum collection, animals were killed and hind-limbs were collected for histology.

FIGS. 3A-3W show histology and microCT on the unilateral ACLT study. Medial tibias stained for toluidine blue and H&E for the (FIG. 3A, 3E) contralateral control (c-control) of the ACLT, (FIG. 3B, 3F) ACLT, (FIG. 3C, 3G) 10W-Veh, and (3D, 3H) 10W-24R,25. Micro-CT images of the medial femurs and tibias for the (FIG. 3I, 3J) c-control of the ACLT, (FIG. 3K, 3M) ACLT, (FIG. 3N, 3O) 10W-Veh, and (FIG. 3P, 3Q) 10W-24R,25. A shadow projection of the femur or the tibia is included in the top left-hand corner of each micro-CT image (FIG. 3I-3Q) with a red line (inside white box) to indicate the location each micro-CT image is taken from within the joint. Histomorphometric analysis of (FIG. 3R) cartilage area, and (FIG. 3S) OARSI scoring in the medial tibias. Micro-CT analysis of the (FIG. 3T, 3U) cartilage volume in medial and lateral tibias in the treated rats and in the (FIG. 3V, 3W) c-control and ACLT rats. Groups with different letters (A or B over bar) denote statistical significance using an alpha=0.05.

FIGS. 4A-4S show serum levels of cytokines, chemokines, and growth factors on post-operative bilateral ACLT day 0 and day 70. Serum was collected from male Sprague Dawley rats that underwent the bilateral ACLT. Factors were measured from animals that received both the Veh and 24R,25 injections. (FIG. 4A-4D) Anti-inflammatory factors involved in OA, (FIG. 4E-4P) pro-inflammatory markers found in OA, (FIG. 4Q, 4R) cytokines and inflammatory factors, and (FIG. 4S) growth factors involved in cartilage remodeling in OA. Groups with different letters (A or B over bar) denote statistical significance using an alpha=0.05.

FIGS. 5A-H show histology, histomorphometrics, and OARSI scoring of the bilateral ACLT rats. Masson's trichrome staining of medial femurs and magnified images for the (FIG. 5A, 5B) vehicle and (FIG. 5C, 5D) 24R,25 treated limbs. Histomorphometric analysis of the (FIG. 5E) articular cartilage area, (FIG. 5F) fibrotic area, and (FIG. 5G) percent healthy subchondral bone. (FIG. 5H) OARSI scoring of OA severity. Groups with different letters denote statistical significance using an alpha=0.05. FT, fibrotic tissue; black asterisk, microfracture; scale bar, 500 μm.

FIGS. 6A-C show stimulation with IL-1β and TNF-α. rArCs were treated with 10 ng/mL IL-1β, or 10 ng/mL of TNF-α or nothing (con) for 24 hours. (FIG. 6A) Total DNA was measured from the cell monolayer and (FIG. 6B) MMP-13, and (FIG. 6C) PGE2 proteins were measured from the conditioned media. Groups not sharing a letter (D, C, B or A over bar) are statistically significant using an alpha=0.05.

FIGS. 7A-7F show assessment of miR-122 and miR-451 transfection or inhibition on TNF-α stimulated rat articular chondrocytes. rArCs were transfected at 60% confluence with miR-122, miR-451, miR-122-inhibitor (122-Inh), miR-451 inhibitor (451-Inh), or an empty vehicle (Lipo) for 24 hours followed by 24 hours with or without 10 ng/mL TNF-α stimulation. (FIG. 7A, 7D) Total DNA was measured from the cell monolayer and (FIG. 7B, 7E) MMP-13 and (FIG. 7C, 7F) PGE2 proteins were measured from the conditioned media. Groups not sharing a letter (A, B or C over bar) are statistically significant using an alpha=0.05.

FIGS. 8A-8F show assessment of crosstalk between WNT/β-catenin and TNF-α pathway. rArCs were treated at 80% confluence with WNT/β-catenin agonist lithium chloride (LiCl) or WNT/β-catenin antagonist XAV-939 (XAV) for 24 hours followed by 24 hours with or without 10 ng/mL TNF-α stimulation. (FIG. 8A, 8D) Total DNA was measured from the cell monolayer and (FIG. 8B, 8E) MMP-13 and (FIG. 8C, 8F) PGE2 proteins were measured from the conditioned media. Groups not sharing a letter (A, B or C over bar) are statistically significant using an alpha=0.05.

FIGS. 9A-9I show assessment of crosstalk between WNT/β-catenin and IL-1β pathway. rArCs were treated at 80% confluence with WNT/β-catenin agonist lithium chloride (LiCl) or WNT/β-catenin antagonists XAV-939 (XAV), or PKF-118 (PKF) for 24 hours followed by 24 hours with or without 10 ng/mL IL-1β stimulation. (FIG. 9A, 9D, 9G) Total DNA was measured from the cell monolayer and (FIG. 9B, 9E, 9H) MMP-13 and (FIG. 9C, 9F, 9I) PGE2 proteins were measured from the conditioned media. Groups not sharing a letter (A, B, C and/or D over bar) are statistically significant using an alpha=0.05. An asterisk denotes significance against the non-IL-1β stimulated control using a student's t-test.

FIG. 10 shows a diagram of inflammatory mediators regulated by miR-122 and/or miR-451.

FIGS. 11A-11B show in vivo experimental designs for miR-451-PI and miR-122-M studies. In the (FIG. 11A) miR-451-PI study, eighteen Sprague Dawley rats underwent a bilateral ACLT surgery to induce OA. Two animals were used as controls (n=4). The eighteen animals were split into two cohorts: 1) a prophylactic cohort that received intra-articular injections of miR-451-PI (n=9) in left hind limbs or NC-PI (n=9) in right hind limbs twice-weekly (red arrows) three days following surgery for a total duration of 6 weeks, and 2) a therapeutic cohort where we allowed OA to develop for 3 weeks before beginning the same injection regimen (n=9 per treatment group, but one animal died resulting in an n=8) for 3 weeks until animals were euthanized on day 42. In the (FIG. 11B) miR-122-M study, eight Sprague Dawley rats underwent a bilateral ACLT surgery to induce OA. Three animals were used as controls (n=6). Animals received injections of NC-M (n=8) or 122-M (n=8) every 9 days for a total of three injections (red arrows). The injection leg was randomized for each animal. Animals were euthanized at the end of 30 days.

FIGS. 12A-12X show gross images, micro-CT, and histology from the prophylactic cohort of the miR-451-PI study. (FIG. 12A, 12C, 12E) Gross images, (FIG. 12B, 12D, 12F) 3D micro-CT rendering, (FIG. 12G-12L) medial and lateral micro-CT images. For FIG. 12G-12L, a shadow projection of the femur is included in the top corner of each micro-CT image with a red line (in white box) to indicate the location each micro-CT image is taken from within the joint. (FIG. 12M-12R) Masson's trichrome histology and the respective zoomed-in images as depicted by the black box (FIG. 12S-12X) for the normal control, NC-PI, and 451-PI groups, respectively. Scale bar: 500 μm. Subchondral bone (sub bone); medial (med); lateral (lat); arrows, areas of cartilage erosion or subchondral bone remodeling; arrowheads, abnormal cartilage remodeling; C, cartilage; FT, fibrotic tissue; asterisk, microfracture.

FIGS. 13A-13H show prophylactic administration of 451-PI decreases OA severity. Histomorphometric analysis of (FIG. 13A) articular cartilage area, (FIG. 13B) fibrotic area, (FIG. 13C) percent healthy subchondral bone, and (FIG. 13D) OARSI scoring. Micro-CT bone volume/total volume (BV/TV) analysis of (FIG. 13E) medial femurs, (FIG. 13F) lateral femurs, (FIG. 13H) total (medial+lateral, FIG. 13G), and the comparison of medial and lateral femurs. An asterisk indicates statistical significance using an alpha equal to 0.05.

FIGS. 14A-14X show gross images, micro-CT, and histology from the therapeutic cohort of the miR-451-PI study. (FIG. 14A, 14C, 14E) Gross image, (FIG. 14B,14D, 14F) 3D micro-CT rendering, (FIG. 14G-14L) medial and lateral micro-CT images. In FIG. 14G-14L, a shadow projection of the femur is included in the top corner of each micro-CT image with a red line (in white box) to indicate the location each micro-CT image is taken from within the joint. (FIG. 14M-14R) Masson's trichrome histology and the respective zoomed-in images as depicted by the black box (FIG. 14S-14X) for the normal control, NC-PI, and 451-PI groups, respectively. Scale bar: 500 μm. Subchondral bone (sub bone); medial (med); lateral (lat); arrows, areas of cartilage erosion or subchondral bone remodeling; arrowheads, abnormal cartilage remodeling; C, cartilage; FT, fibrotic tissue; asterisk, microfracture.

FIGS. 15A-H show therapeutic administration of 451-PI does not decrease OA severity. Histomorphometric analysis of (FIG. 15A) articular cartilage area, (FIG. 15B) fibrotic area, (FIG. 15C) percent healthy subchondral bone, and (FIG. 15D) OARSI scoring. Micro-CT bone volume/total volume (BV/TV) analysis of (FIG. 15E) medial femurs, (FIG. 15F) lateral femurs, (FIG. 15G) total (medial+lateral), and (FIG. 15H) the comparison of medial and lateral femurs. An asterisk indicates statistical significance using an alpha equal to 0.05.

FIGS. 16A-16F show maximum twitch and tetany of the tibialis anterior muscle are not affected in OA. (FIG. 16A, 16C, 16E) Maximum twitch and (FIG. 16B, 16D, 16F) maximum tetany of animals in the prophylactic cohort, therapeutic cohort, and the 122-M study, respectively. An asterisk indicates statistical significance using an alpha equal to 0.05.

FIGS. 17A-17X show gross images, micro-CT, and histology from the miR-122-M study. (FIG. 17A, 17C, 17E) Gross image, (FIG. 17B, 17D, 17F) 3D micro-CT rendering, (FIG. 17G-17L) medial and lateral micro-CT images. In FIG. 17G-17L, a shadow projection of the femur is included in the top corner of each micro-CT image with a red line (in white box) to indicate the location each micro-CT image is taken from within the joint. (FIG. 17M-17R) Masson's trichrome histology and the respective zoomed-in images as depicted by the black box (FIG. 17S-17X) for the normal control, NC-PI, and 451-PI groups, respectively. Scale bar: 500 μm. Subchondral bone (sub bone); medial (med); lateral (Lat); arrows, areas of cartilage erosion or subchondral bone remodeling; arrowheads, abnormal cartilage remodeling; C, cartilage; FT, fibrotic tissue; asterisk, microfracture.

FIGS. 18A-18H show administration of 122-M does not decrease OA severity. Histomorphometric analysis of (FIG. 18A) articular cartilage area, (FIG. 18B) fibrotic area, (FIG. 18C) percent healthy subchondral bone, and (FIG. 18D) OARSI scoring. Micro-CT bone volume/total volume (BV/TV) analysis of (FIG. 18E) medial femurs, (FIG. 18F) lateral femurs, (FIG. 18G) total (medial+lateral), and the comparison of (FIG. 18H) medial and lateral femurs. An asterisk indicates statistical significance using an alpha equal to 0.05.

### DETAILED DESCRIPTION

The present disclosure describes compositions and methods of treating the complex and multifaceted disease of OA. Compositions of the invention intervene in the catabolic biochemical signaling axis caused by over-modulation of inflammatory molecules that cause OA. The compositions and methods for their use promote chondrocyte proliferation and reduce the effects of IL-1β in vitro and are effective therapeutic agents in vivo for the treatment of OA.

One embodiment of the invention is a pharmaceutical composition for use as an inhibitor of osteoarthritis comprising an miR-122, an miR-122 mimic, an inhibitor of microRNA-451 (miR-451) or any combination of these agents. In one embodiment, the miR-451 inhibitor is rno-miR-451-5p miRCURY LNA miRNA-inhibitor. In another embodiment, the agent is an miR-122 mimic, such as rno-miR-122 mimic 2.0. The pharmaceutical composition can be administered to a subject to inhibit an inflammatory response, and more particularly to inhibit an inflammatory response in articular chondrocytes of the cartilage of a joint. Delivery of the pharmaceutical composition a therapeutically sufficient amount of the pharmaceutical composition comprising any of an miR-122, an miR-122, and/or an inhibitor of miR-451 to a joint provides a method of treating or inhibiting OA or OA disease progression in a subject in need thereof. The therapeutically sufficient amount of the pharmaceutical composition is a dose and dosing regimen able to inhibit an inflammatory response, particularly in cells of a joint, such as articular chondrocytes and other cells associated with the region of a bone joint, including immune cells, macrophages and other motile cells that may enter a joint. A joint that has been injured or received other trauma may be particularly at risk of infiltration by immune cells that exacerbate the inflammatory response in the region of the joint.

The pharmaceutical composition may be administered by injection near or directly into a joint suspected of or known to be affected by OA. A treatment regimen for administration of the pharmaceutical composition may be a single prophylactic dose, particularly following an acute injury to at least one joint, or administration may be sustained for days or weeks at suitable intervals. Suitable intervals may be dependent upon the extent of an injury and may be determined by a knowledgeable practitioner to be multiple times daily, twice daily, once daily, twice weekly, once weekly, and repeated for many days or weeks or any other dosing regimen determined to be suitable for treating an individual subject. In one embodiment, the pharmaceutical composition is administered at least twice a week for at least six weeks. In one embodiment, the pharmaceutical composition further comprises a tag that allows homing to the articular cartilage or joint space, thus allowing for systemic administration.

The invention is based on the discovery of the therapeutic effect of inhibiting the dysregulated inflammatory pathways of IL-1β and TNF-α that control both the chronic inflammatory responses as well as the production of catabolic matrix-degrading enzymes for the treatment of OA. Embodiments are provided that intervene in the chronic inflammatory signaling that underlies OA, targeting the disease at its root to prevent OA progression. Thus, in one embodiment, the pharmaceutical composition comprises an inhibitor of miR-451 that modulates the miR-451-mediated inflammatory processes of osteoarthritis disease progression.

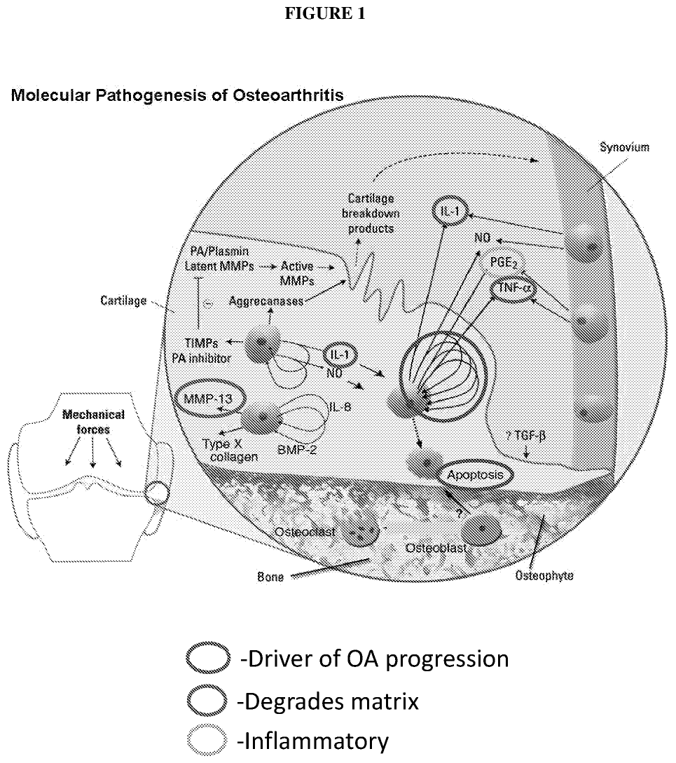

FIG. 1 illustrates the molecular pathogenesis of osteoarthritis that leads to destruction of cartilage within a joint. The inflammatory milieu caused by IL-1β and TNF-α stimulation in the articular chondrocytes may further involve other drivers of OA progression and inflammatory cytokines, including TNF-α, NO, IL-8, MMP-13, BMP-2, TGF-β, and PGE₂. In addition, dysregulated inflammatory cytokines and chemokines may include M-CSF, GM-CSF, G-CSF, GRO/KC. IFN-γ, MIP-1α, VEGF, and interleukins such as IL-1α, IL-2, IL-4, IL-5, IL-6, IL-7, IL-IL-12(p70), IL-17 and IL-18.

In another embodiment, the pharmaceutical composition comprises miR-122 or an miR-122 mimic, which is administered to an affected joint where it also inhibits the inflammatory response in articular chondrocytes and other cells associated with the cartilaginous structures within the joint. The miR-451 inhibitor, miR-122 or miR-122 mimic or any combination of these agents may be packaged with antisense-oligonucleotides, lipid nanoparticles or other means of delivery to chondrocytes of joint suspected of being affected or known to be affected by the risk of OA.

A unilateral and bilateral ACLT model of OA was first established to evaluate different therapeutics, as will be disclosed in the Examples of the invention. This was essential for creating and understanding a specific model of OA. The therapeutic potential of 24R,25 treatment was tested in these two models of OA. While 24R,25 treatments can be effective in a mild model of OA, treatment with 24R,25 in a severe model was not able to mitigate OA progression. Thus, miRs, miR mimics or miR inhibitors were tested as therapeutics for the treatment of OA, since miR-122 maintains the resting zone chondrocytes and miR-451 drives terminal differentiation, making each a viable target for the aberrant signaling a stimulation and prevent the production of downstream targets in primary rat articular chondrocytes. Interestingly, when evaluated in the severe bilateral model of OA in Sprague Dawley rats, protective effects of miR-122 treatment on OA were not detected. However, inflammatory processes were mitigated in the articular chondrocytes and suggest that greater doses and/or a longer term of treatment will be effective in inhibiting development of OA. Over the last couple of decades, years of research have gone into therapeutic oligonucleotides, but only a handful have received market approval. The most recent and well-known examples are the new immerging mRNA-bases vaccines for the ZIKA, EBOLA, and SARS-CoV2 viruses. These lend value to the beneficial applications of oligonucleotide therapies despite the current limitations with local administration, liver accumulation, and rapid destruction. These examples provide evidence that miR-122 is acting through a means that can target both IL-1β and TNF-α signaling pathways, even though the precise mechanism of action remains unclear. While miR-122 mimic administration was not able to mitigate OA progression in vivo, the robust in vitro data indicate that this microRNA can be an effective anti-inflammatory therapeutic in articular chondrocytes with a dosing concentration and/or dosing frequency.

The results with miR-451 were the most surprising. Prophylactic administration of the miR-451 inhibitor resulted in a decreased OA severity in the severe bilateral OA model. This microRNA was originally chosen as a negative control but was found to be a surprisingly effective as a treatment. Interestingly, miR-451 transfection exacerbated the IL-1β stimulated increase in cytokines, chemokines, and catabolic enzymes even more so than IL-1β stimulation alone. However, miR-451 transfection had no effect on these same molecules following TNF-α stimulation. These data indicate that miR-451 is potentially targeting an anti-inflammatory molecule that interferes with the IL-1β signaling pathway, but not the TNF-α signaling pathway. Additionally, miR-451 expression was elevated in the cartilage of OA in Sprague Dawley rats, indicating that inhibiting miR-451 is a target for alleviating OA. When we tested prophylactic administration of miR-451 inhibitor in the severe bilateral model of OA in Sprague Dawley rats, there was a significant decrease in OA severity compared to vehicle injections. These results were after only twice-weekly injections for a duration of 6 weeks. This frequency was determined based on a previously published protocol for a different microRNA. This dosing regimen (dosing concentration or frequency) can be altered to achieve an even more robust response or decreased to make miR-451 administration cost-optimal and clinically scalable. A dosing experiment where miR-451 is tagged with a fluorophore to determine the duration miR-451 persists in the joint space is contemplated.

While prophylactic administration of miR-451 inhibitor was efficacious, therapeutic administration after OA had already developed did not reduce or reverse OA severity. This may be due to the injection duration (3 total weeks of therapeutic injections as compared to 6 weeks), or to the fact that reversing OA after it has developed has never been achieved to date. A longer duration of 451-PI treatment after OA is contemplated and may achieve the desired therapeutic effects of miR-45-PIs.

As used herein, the term “miR-451-5p inhibitor” is used to refer to any molecule that can inhibit the effects of native or mimic miR-451-5p, either through a partial or complete binding to the miR-451-5p mimic sequence. A traditional inhibitor will have a complimentary sequence to the miR-451-5p mimic sequence, either partially or fully. Since there is cross-homogeneity with different species, i.e., rno-miR-451-5p is the same as mu-miR-451-5p, miRNAs from one species may be administered to a subject of another species.

The effects of the miR-122-5p mature mimic sequence (SEQ ID NO:2) may also be replicated using sequences that include any possible point-mutations, such as those listed below, either alone or in combination.

TABLE 1
miRNA Sequences and Examples of Mutated, Mimic or Inhibitor Sequences. All sequences shown
5′-3′, bold and underlined nucleotides are mutations of the native/mimic sequences.

| Name | Sequences | SEQ ID reference |
|---|---|---|
| microRNA-122-5p | UGG AGU GUG ACA AUG GUG UUU G | SEQ ID NO: 1 |
| mature mimic | | |
| (miR-122) | | |
| Mutated miR-122 | AGG AGU GUG ACA AUG GUG UUU G | SEQ ID NO: 2 |
| Mutated miR-122 | UCG AGU GUG ACA AUG GUG UUU G | SEQ ID NO: 3 |
| Mutated miR-122 | UCC AGU GUG ACA AUG GUG UUU G | SEQ ID NO: 4 |
| Mutated miR-122 | UCG UGU GUG ACA AUG GUG UUU G | SEQ ID NO: 5 |
| Mutated miR-122 | UCG AAU GUG ACA AUG GUG UUU G | SEQ ID NO: 6 |
| Mutated miR-122 | UCG AGU GUG UUG AUG GUG UUU G | SEQ ID NO: 7 |
| Mutated miR-122 | UCG AGU GUG ACA AUG GUG AAA G | SEQ ID NO: 8 |
| Mutated miR-122 | UCG AGU GUG ACA GCG GUG UUU G | SEQ ID NO: 9 |
| microRNA-451-5p | AAA CCG UUA CCA UUA CUG AGU U | SEQ ID NO: 10 |
| mature mimic sequence | | |
| (miR-451) | | |
| miR-451 inhibitor | AGT AAG GGT AAC GGT T | SEQ ID NO: 11 |
| miR-451 inhibitor | -GT AAT GGT AAC GGT T | SEQ ID NO: 12 |
| miR-451 inhibitor | UCA ACA UCA GUC UGA UAA GCU A | SEQ ID NO: 13 |

The invention encompasses any other mutated miR-122 mimics and any other miR-451 inhibitor sequences that can be made with various substitutions. The inhibitors bind to the native miR-451 when administered to a subject. For example, the native miR-451 of SEQ ID NO:10 will be bound with the example inhibitor of SEQ ID NO:11 in the alignment shown in Table 2:

TABLE 2
Example of Binding between miR-451
Mimic Sequences (SEQ ID NO: 10)
and Sequences of an miR-451
Inhibitor (SEQ ID NO: 11).

| | | | | | | | | | | | | | | | | | | | | | | | |
|---|---|---|---|---|---|---|---|---|---|---|---|---|---|---|---|---|---|---|---|---|---|---|---|
| Mimic: | A | A | A | C | C | G | U | U | A | C | C | A | U | U | A | C | U | G | A | G | U | U | 3′ |
| 5′ | | | | | | | | | | | | | | | | | | | | | | | |
| Inhibitor: | — | T | T | G | G | C | A | A | T | G | G | | A | A | T | G | A | — | — | — | — | — | 5′ |
| 3′ | | | | | | | | | | | | | | | | | | | | | | | |
| | | | | | | | | | | | | G | | | | | | | | | | | |

In the example of binding to an inhibitor sequence as shown in Table 2, there is a binding location where the inhibitor does not bind to the mimic sequence (G). This type of modification can be made in any location and still elicit inhibitor effects. A longer inhibitor or shorter inhibitor would also prevent the mimic from acting. Additionally, the inhibitor can be made as an RNA inhibitor (contains U) or as a DNA inhibitor (contains T) with modifications to the backbone. Modifications to the backbone of the inhibitor such as locked-nucleic acid backbones and or fully phosphorothioate (PS) modified backbones all function as inhibitors to this mimic and can increase the stability and longevity of the inhibitor but accomplish the same end result.

Once a patient is identified being at an elevated risk of OA, suitable clinical intervention can be undertaken to prophylactically treat to inhibit, lessen the effects of, or even prevent OA from developing or progressing. A patient or subject to be treated by any of the methods of the present disclosure can mean either a human or a non-human animal including, but not limited to dogs, horses, cats, rabbits, gerbils, hamsters, rodents, birds, aquatic mammals, cattle, pigs, camelids, and other zoological animals. As discussed above, the miRs of the invention are conserved and therefore have a high degree of homology between many species.

In some embodiments, the treatment is administered to the subject in a therapeutically effective amount. By a “therapeutically effective amount” is meant a sufficient amount of active agent to decrease the likelihood of OA at a reasonable benefit/risk ratio applicable to any medical treatment. In some aspects, OA is inhibited or prevented and the effects of OA disease progression are mitigated.

In some aspects, the invention provides nucleic acid sequences that encode the miRs of the invention. Further, the invention comprehends vectors which contain or house coding sequences for the miRs. Examples of suitable vectors include but are not limited to plasmids, cosmids, viral based vectors, expression vectors, etc. Vectors comprising the miRs of the invention may be produced by any suitable method, many of which are known to those of skill in the art. For example, they may be chemically synthesized, or produced using recombinant DNA technology (e.g., in bacterial cells, in cell culture (mammalian, yeast or insect cells), in plants or plant cells, or by cell-free prokaryotic or eukaryotic-based expression systems, by other in vitro systems, etc.).

The compositions are generally administered in a pharmaceutically acceptable formulation which includes suitable excipients, elixirs, binders, and the like (generally referred to as “pharmaceutically and physiologically acceptable carriers”), which are pharmaceutically acceptable and compatible with the active ingredients. These may include pharmaceutically acceptable salts (e.g., alkali metal salts such as sodium, potassium, calcium or lithium salts, ammonium, etc.) or as other complexes. It should be understood that the pharmaceutically acceptable formulations include solid, semi-solid, and liquid materials conventionally utilized to prepare solid, semi-solid and liquid dosage forms such as tablets, capsules, liquids, aerosolized dosage forms, and various injectable forms (e.g., forms for intravenous administration), etc. Water may be used as the carrier for the preparation of compositions which may also include conventional buffers and agents to render the composition isotonic. The composition of the present disclosure may contain any such additional ingredients so as to provide the composition in a form suitable for the intended route of administration. In addition, the composition may contain minor amounts of auxiliary substances such as wetting or emulsifying agents, pH buffering agents, and the like. Similarly, the carrier or diluent may include binders or encapsulants (lactose, liposomes, etc.). It is expected that the active components will be present at about 1% to about 99% of the composition and the vehicular “carrier” will constitute about 1% to about 99% of the composition. The pharmaceutical compositions of the present disclosure may include any suitable pharmaceutically acceptable additives or adjuncts to the extent that they do not hinder or interfere with the therapeutic effect(s) of the composition.

The compositions (preparations) of the present disclosure are formulated for administration by any of the many suitable means which are known to those of skill in the art, including but not limited to orally, by injection, topically, transdermally, by inhalation of an aerosol, by microneedle delivery, etc. In some aspects, the mode of administration is by injection or intravenously.

The administration of the compound of the present disclosure may be intermittent, or at a gradual or continuous, constant or controlled rate (e.g., in a sustained release formulation which further extends the time of bioavailability). In addition, the time of day and the number of times per day that the pharmaceutical formulation is administered may vary and are best determined by a skilled practitioner such as a physician.

Administration of the compound by any means may be carried out as a single mode of therapy, or in conjunction with other therapies and treatment modalities, e.g., antibiotics, pain medication, gene therapy, etc. “In conjunction with” refers to both administration of a separate preparation of the one or more additional agents, and also to inclusion of the one or more additional agents in a composition of the present disclosure.

As used herein, “increase” or “decrease” refers to increasing or lowering by, for example, at least about 5%, 10%, 20%, 30%, 40%, 50%, 60%, 70%, 80%, 90%, or more when compared to at least one type of standard or control.

The terms “sensitivity” and “specificity” are used herein to refer to statistical measures of the performance of diagnostics tests. Sensitivity refers to a proportion of positive results which are correctly identified by a test. Specificity measures a proportion of the negative results that are correctly identified by a test. The term “high specificity” refers to specificity that is equal to or over 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. The term “high sensitivity” refers to sensitivity that is equal to or over 80%, 81%, 82%, 83%, 84%, 85%, 86%, 87%, 88%, 89%, 90%, 91%, 92%, 93%, 94%, 95%, 96%, 97%, 98%, or 99%.

Before exemplary embodiments of the present invention are described in greater detail, it is to be understood that this invention is not limited to particular embodiments described, and as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLES OF THE INVENTION

The following Examples of the invention provide an animal model of OA and demonstrate the therapeutic potential of inhibiting the dysregulated inflammatory pathways of IL-1β and TNF-α for the treatment of OA. The Examples demonstrate the development of an animal model of severe OA and evaluate the role of the vitamin D metabolite 24R,25 and its chondroprotective effects in the presence of inflammation. Additional Examples demonstrate the administration of specific microRNAs, miR-122 and miR-451, which are regulatory molecules that have overarching control of gene circuits that are often dysregulated in OA.

The Examples also demonstrate the effects of treating with miR-122 or inhibiting miR-451 in vivo. The bilateral ACLT model of OA was designed to examine the prophylactic and therapeutic potential of inhibiting miR-451 as well as the prophylactic potential of miR-122 mimic by evaluating treatment at the onset of joint trauma and treatment after OA has developed.

### Example 1

Treatment of Unilateral or Bilateral Model of OA with 24R,25-Dihydroxyvitamin D3

### Rationale and Overview

The goal of this Example was three-fold: 1) to develop an ACLT model of OA in house, 2) to evaluate the therapeutic effects of 24R,25 treatment in this model for a 10-week treatment duration, and 3) determine if we could prevent OA from developing if we interrupted that initial inflammatory signaling for the first 5 weeks after trauma and ceased treatments for the remaining 5 weeks. We used a unilateral and bilateral ACLT model to examine the therapeutic effects of 24R,25 over 10 weeks. To develop the animal model of severe OA, rats received ACLT of both hind limbs. The presence of OA was established by assessing serum and histological changes. The changes in many serum cytokines, chemokines, and growth factors from day 0 (before surgery) compared to day 70 (terminal) were robust. While studies have found elevated serum cytokines in humans and some animal models have analyzed these, there are no previous studies that compare this breath of serum markers from day 0 to after OA development in animal models to date. Additionally, the sheer number of elevated molecules lent credence to the systemic response seen due to trauma and OA progression, even 70 days after the initial trauma. The cartilage lesions and the presence of fibrotic tissue as well as mild subchondral bone remodeling observed confirmed the presence of OA in our severe model.

### Materials and Methods

### Animal Study Methodology

A total of 54 skeletally mature male Sprague-Dawley rats (Envigo, Indianapolis, IN), 9-10 weeks of age, weighing 250-300 g at the study onset, were used for these experiments. All animal procedures were performed according to the National Research Council's Guide for the Care and Use of Laboratory Animals guidelines and approved by Virginia Commonwealth University's Institutional Animal Care and Use Committee. All rats were given ad libitum access to standard pellet and water. Based on a previous study (Scott, supra) using a 30% effect size, 20% variance, and a power of 80%, a two-tail analysis indicated an n=8 per group was necessary to yield statistical significance.

### Mild Model of OA Using a Unilateral Anterior Cruciate Ligament Transection

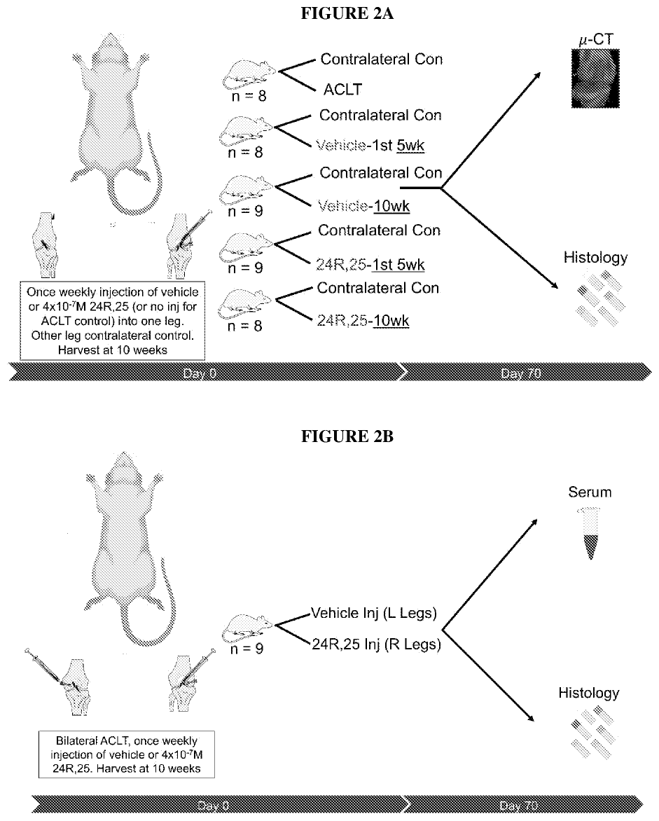

The unilateral ACLT study initially included 45 rats. The experimental design for the unilateral ACLT study is shown in FIG. 2A. Each treatment group had n=8 plus 1 extra animal in case of morbidity or mortality. Three rats died resulting in a total of 42 rats in the study. Rats were sedated with 2-4% isoflurane anesthesia in 400 mL/min 02 to effect. Unilateral anterior cruciate ligament transections (ACLT, animals=45) were performed by medially translocating the patellar tendon in order to visualize and transect the ACL to create a severe model of OA. Left legs served at contralateral controls (c-control) while right legs received either 50 μL of vehicle injections or 4×10⁻⁷ M 24R,25(OH)₂D₃ (24R,2S) injections once a week. Eighteen rats were used to examine a 5-week prophylactic treatment duration while another eighteen rats were used to examine a 10-week prophylactic treatment duration. Nine animals received the unilateral ACLT and did not receive treatments (denoted ACLT). However, 3 animals died in this study resulting in an n=8 for the ACLT rats, for the 5-week vehicle (5W-Veh) treated rats, and the 10-week 24R,25 (10W-24R,25) treated rats. Animals were killed after 70 days. Hind-limbs were collected and micro-CT and histological analyses were performed.

### Severe Model of OA Using a Bilateral Anterior Cruciate Ligament Transection

The second study created a more severe model of OA by performing bilateral ACLTs, using the experimental design shown in FIG. 2B. Treatment groups had n=8 plus 1 extra animal in case of morbidity or mortality. Nine animals were sedated using 2-4% isoflurane anesthesia in 400 mL/min O2 to effect. Bilateral anterior cruciate ligament transections were performed by medially translocating the patellar tendon in order to visualize and transect the ACL. Left legs received vehicle injections and right legs received 24R,25 injections once weekly using the same injection regimen outlined for the previous study. Animals were killed 70 days post-ACLT, and serum and hind-limbs were collected.

### Intra-Articular Injections of 24R,25(OH)2D3

24R,25(OH)₂D₃ was obtained from Enzo Life Sciences (Plymouth Meeting, PA) and dissolved in ethanol at a stock concentration of 10⁻⁴ M in accordance with a previously established protocol (see Boyan et al. PLoS One. 2016; 11(8):e0161782). 40 μL of stock 24R,25 was dissolved in 10 mL sterile 1×PBS to create a final treatment concentration of 4×10⁻⁷ M 24R,25. All animals received injections of 25 μL of 4×10⁻⁷ M 24R,25 in right legs or vehicle (40 μL ethanol dissolved in sterile 1×PBS to a final concentration of 0.4%) in left legs once a week for 70 days based on a previously established protocol (see Boyan et al., supra).

### Blood Serum Collection

Blood was collected the day before surgery (day 0) via tail vein extraction using a 22G Terumo butterfly catheter (Fisher Scientific). Terminal blood (day 70) was collected via cardiac puncture. Rats were euthanized via exsanguination of the abdominal aorta followed by cervical dislocation. All blood was collected under isoflurane anesthesia and approximately 800 mL of blood was collected for each time point. Blood was allowed to clot at room temperature for 30 mins, spun at 1,000×g for 15 mins at 4° C., and serum was collected and snap-frozen for later analysis.

### ELISA Analysis of Serum Samples

Levels of inflammatory molecules commonly associated with OA (Bio-Plex Pro™ Rat Cytokine 23-Plex Assay, Bio-Rad, Hercules, CA) were measured using a magnetic-bead-based assay on a Bio-Plex MAGPIX™ multiplex reader (Bio-Rad) following the manufacture's protocol.

### MicroCT

Samples were fixed in 10% buffered formalin for a minimum of 3 days, excessive soft tissue was removed. Since soft tissue such as cartilage does not normally attenuate with micro-CT, the ionic iodinated-based contrast agent hexabrix 320 (Mallinckrodt Inc., St. Louis, MO, USA) was implemented. Samples were incubated in a 40% hexabrix in 1×PBS in the dark for 1 hour at 37° C. and then scanned. All scans were performed using a resolution of 2240×2240 (image pixel size of 7.91 μm), 45 kVp, 177 μA, 1300 ms exposure time, and rotation step size of 0.35°. After scanning, hexabrix was removed from samples by incubating samples in 1×PBS overnight at 4° C. MicroCT for the unilateral ACLT study resulted in an n size of n=6 for the contralateral control, n=6 for the ACLT, n=7 for 10W-Veh, and an n=6 for the 10W-24R25.

### Histology

Samples were decalcified for 7 days using Decal™ (StatLab Medical Products, Mckinney, TX), rinsed in running DI water for 15 mins, dehydrated in a series of 70-100% ethanol and xylene washes, and embedded with Richard-Allan Scientific Histoplast Paraffin (VWR, Radnor, PA). 5 μm sections were cut every 100-200 μm throughout the joint using a manual microtome (Shandon Finesse 325, Thermo Scientific) and stained using hematoxylin and eosin (H&E), toluidine blue, or Masson's trichrome. Samples were imaged using Zen 2012 Blue Edition software with an AxioCam MRc5 camera and Axio Observer Z.1 microscope (Carl Zeiss Microscopy, Oberkochen, Germany).

### Histomorphometrics

The articular cartilage area, fibrotic area, and percent healthy subchondral bone were measured using Zen 2012 Blue Edition software. For the unilateral ACLT study: the articular cartilage area was measured on 2-3 slides per sample and the average area for each sample was reported for medial tibias. Histomorphometric analysis for the unilateral ACLT study resulted in n=7 for the ACLT group, n=9 for the 10W-Veh group, and n=6 for the 10W-24R,25 group due to histological processing errors. For the bilateral ACLT study, articular cartilage area, fibrotic area, and the percent healthy subchondral bone were measured on one slide per sample for medial femurs. The subchondral bone plate was measured and subdivided into healthy regions (normal cartilage above) and unhealthy regions (subchondral bone remodeling and osteophyte formation, cartilage erosion/delamination). The percent healthy subchondral bone was reported as healthy subchondral bone divided by total subchondral bone*100. Histomorphometric analysis for the bilateral ACLT study resulted in n=6 for the vehicle group, and n=7 for the 24R,25 group due to histological processing errors.

### OARSI Scoring

Two blinded reviewers scored histological samples and assigned a grade (0-6.5) to assess OA depth progression into the cartilage and a stage (0-4) to assess the horizontal extent of cartilage involvement along the articulating surface according to the OARSI scoring system guidelines (see Pritzker et al. Osteoarthritis Cartilage. 2006 January; 14(1):13-29). The grade and stage were multiplied together to obtain the OARSI score. The higher the score, the more severe OA present, with a max possible score of 26. OARSI scoring was based on the histology and the same n number was used for each group as reported above.

### Statistical Analysis

Data are represented as mean±standard error. Groups with different letters are statistically significant using a one-way analysis of variance followed by a two-tailed Tukey correction using an alpha=0.05. Blood serum of day 0 vs day 70 was analyzed used a two-tailed paired t-test using an alpha=0.05. All statistical analyses were performed using GraphPad Prism version 5.04 or JMP Pro 14.

### Results

### 24R,25 Treatment in the Mild Model of OA

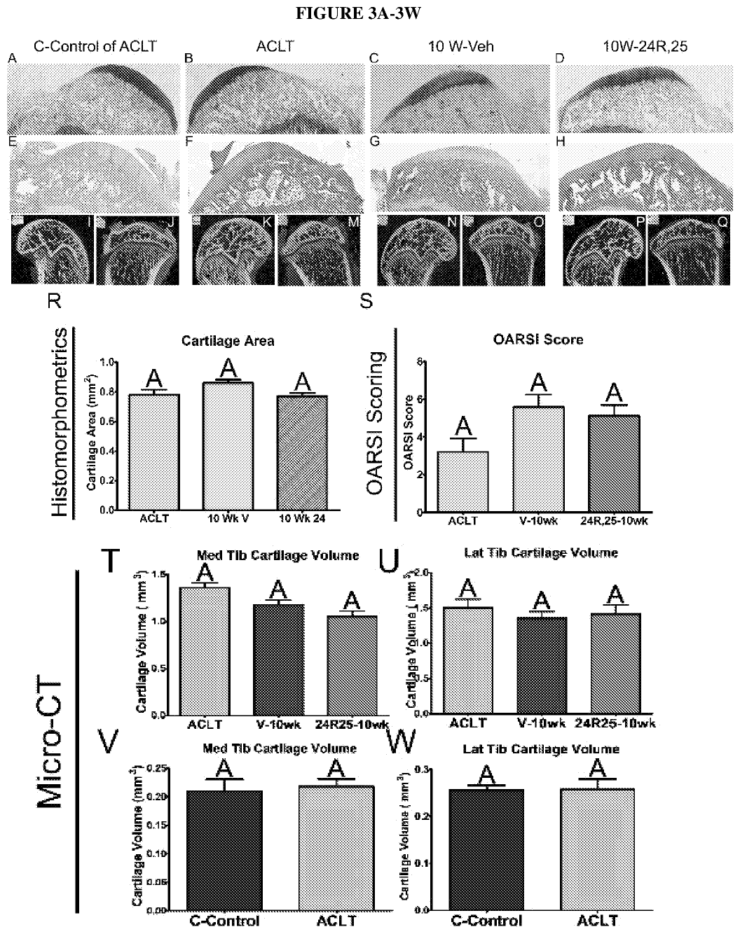

Toluidine blue and H&E staining were performed on medial tibias and quantified, as illustrated by representative sections and graphs in FIGS. 3A-3W. Shown are the c-control for the ACLT group (FIG. 3A, 3E), the ACLT limb (FIG. 3B, 3F), the 10W-Veh limb (FIG. 3C, 3G), and the 10W-24R,25 limb (FIG. 3D, 3H). Qualitatively, toluidine blue revealed ample presence of glycosaminoglycans in all treatment groups and their c-controls (FIG. 3A-3D). H&E staining revealed a smooth articular cartilage surface and intact subchondral bone plate (FIG. 3E-3H). No evidence of cartilage erosion or degradation was seen in any group. Qualitative micro-CT of medial femurs and tibias showed no evidence of OA and no subchondral bone erosion or remodeling for any group (FIG. 3I-3Q).

To assess 24R,25 as a treatment, we first examined the 10W treatments of 24R,25, or Veh vs the ACLT alone. There was no difference in cartilage area as determined by histomorphometric analysis (FIG. 3R). The OARSI score corroborated these results (FIG. 3S). An OARSI score between 3 and 12 indicates an early-stage osteoarthritic lesion. The mean OARSI scores for the ACLT, 10W-Veh, and 10W-24R,25 were 3.19, 5.56, and 5.11 respectively. Micro-CT analysis of the cartilage volume on the medial and lateral tibia found no difference in cartilage volume between groups (FIG. 3T, 3U). Finally, to determine if OA was produced in this model, cartilage volume was compared between the c-control to its respective ACLT limb (FIG. 3V, 3W). No difference was found in the medial or lateral tibias by micro-CT quantification (FIG. 3V, 3W).

### 24R,25 Treatment in a Severe Model of OA

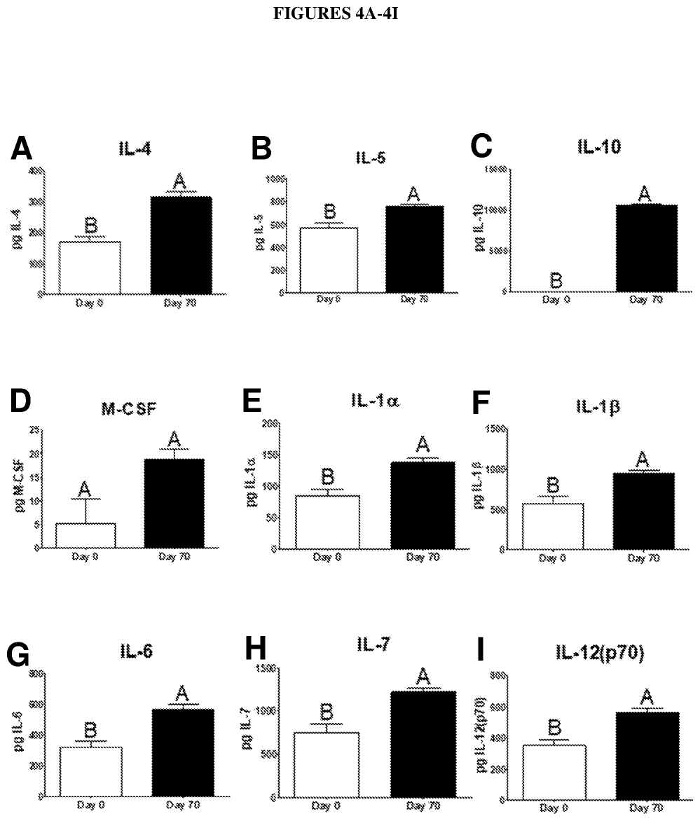

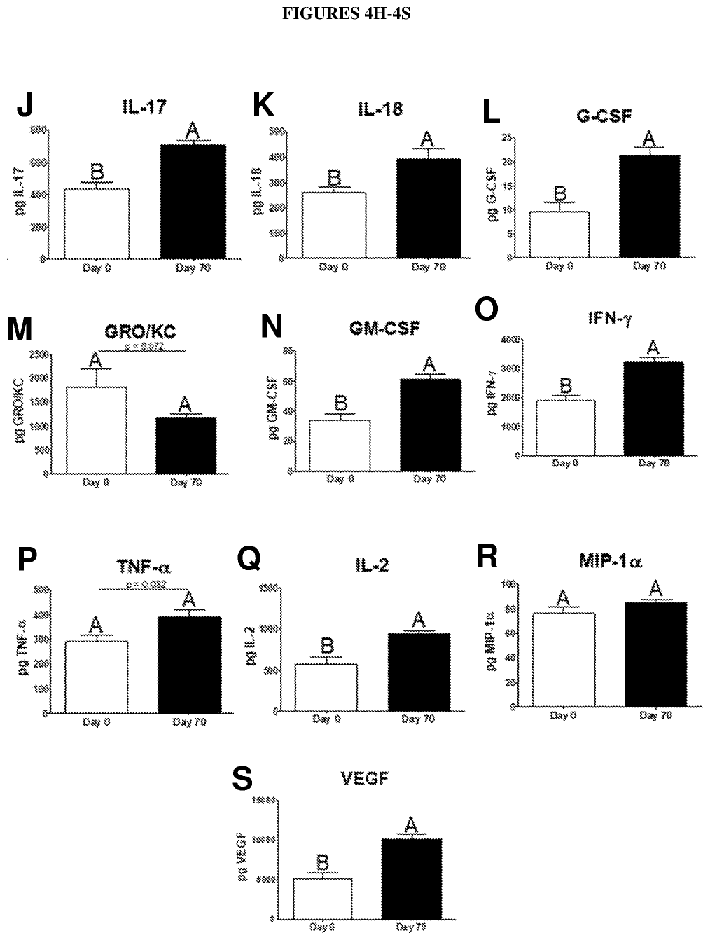

To investigate the effects of 24R,25 in a severe OA model, injections were given once weekly following a bilateral ACLT surgery (first dose on the day of surgery) for a duration of 10 weeks since elevated serum ad synovial fluid cytokines, chemokines, and growth factors have been found elevated in OA in other studies. Serum was analyzed from day 0 (before surgery) and compared to serum on day 70, with results shown in FIGS. 4A-4S. All cytokines, chemokines, and growth factors associated with OA were elevated on day 70 compared to day 0, with the exception of GRO/KC, M-CSF, MIP-1α, or TNF-α.

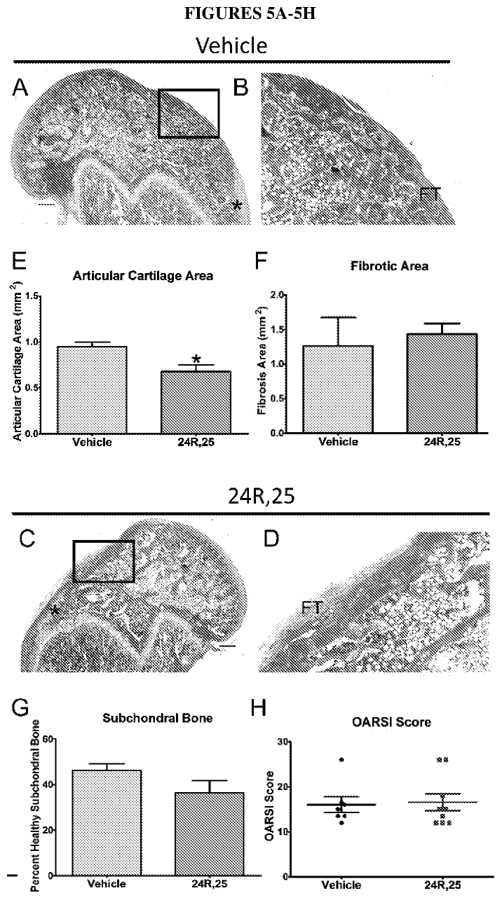

Medial femurs were stained with Masson's trichrome, shown in FIGS. 5A-5D. Evidence of cartilage fibrillation and microfractures (asterisk) in the subchondral bone plate was noted in both groups (FIG. 5A, 5C). At higher magnification, chondrocyte hypertrophy, vertical fissures, and fibrotic tissue (FT) formation for both vehicle and 24R,25 treated limbs were seen (FIG. 5B, 5D).

Histomorphometric analysis of medial femurs showed 24R,25 limbs had significantly less articular cartilage area compared to vehicle limbs (FIG. 5E). However, there was an equivalent amount of fibrotic area and subchondral bone (FIG. 5F, 5G). An OARSI score between 12 and 18 indicates a mid-stage osteoarthritic lesion. Both Veh and 24R,25 had an average score range of 16.07 and 16.61 respectively with no difference between treatment limbs (FIG. 5H).

Previous work in our lab showed that treatments with 24R,25 mitigated OA progression in a unilateral ACLT model of OA four weeks following ACLT surgery. However, this model was outsourced and wasn't established in house. This Example 1) established an ACLT OA model in house, and 2) evaluated two different treatment timelines.

With inflammatory molecules being elevated>1-year post-traumatic injury, inflammatory signaling plays a pivotal role in OA disease development. 24R,25 is able to prevent IL-1β stimulated increases in MMP-13, PGE2, and NO. We hypothesized that inhibiting these inflammatory pathways in the early stages of OA development would stop the positive feedback loop and prevent OA progression even after treatments stopped.

To this end, our unilateral ACLT study included a group that received either Veh or 24R,25 injections for 5 weeks followed by 5 weeks of no treatment. The other cohort in the unilateral ACLT study received injections for the entire 10-week duration of the study. However, when analyzing the 10-week treatment limbs, no differences between the ACLT group, 10W-Veh, or 10W-24R,25 treated limbs were found in either the histomorphometric articular cartilage area or in the micro-CT analysis of the articular cartilage volume. The smooth articulating cartilage surface in histological specimens and lack of differences in the micro-CT analysis led us to question if OA was present in this model. Micro-CT analysis of the contra-lateral control compared to the no-treatment ACLT leg indicated osteoarthritic lesions were not present. Due to the lack of evidence indicating OA was present in this model, no 5W groups were analyzed.

The results of the mild unilateral ACLT model to produce OA led us to develop a bilateral ACLT model to produce a more severe OA phenotype. We confirmed OA induction in the bilateral model through serum, histomorphometric analysis, and OARSI scoring.

Many pro-inflammatory, anti-inflammatory, and growth factors are shown to be elevated in the synovial fluid, synovial membrane, and subchondral bone and cartilage of OA patients; and elevation of TNF-α, IL-1, and IL-6 in serum has been shown in obesity and positively correlated to obesity-related OA. The identification of serum markers as a prognostic and diagnostic tool is on the rise for detecting early OA before radiographic evidence. One study by Ling and colleagues found elevated serum IL-1α, IL-2, IL-15, GM-CSF, MMP-7, and MIP-1α compared with non-OA controls (Osteoarthr Cartil. 2009 January; 17(1):43-8). Other studies have found elevated serum cytokines including IL-1β, IL-4, IL-5, IL-6, IL-12, IL-18, MIP-1α, IFN-γ, VEGF. Interestingly, elevation of the anti-inflammatory serum cytokines IL-4, IL-5, and IL-10 are hypothesized to increase in OA in a compensatory mechanism to help control levels of pro-inflammatory cytokines. To date, no other studies have examined this magnitude of elevated serum cytokines, chemokines, and growth factors before and after OA onset in an ACLT rat model of OA.

Interestingly, histomorphometric analysis of 24R,25 treated limbs in the bilateral study showed a decrease in articular cartilage area compared to Veh treated limbs. No other indication of differences in OA severity between treatment groups could be seen, whether in qualitative histology, histomorphometric analysis of the fibrotic area and healthy subchondral bone, or in the OARSI scoring. The OARSI score indicates this is a mid-stage model of OA with a mean OARSI score of 15 for both groups. Based on our previous work, it is clear that treatment with 24R,25 is able to mitigate OA progression in a mild model of OA (see Boyan et al. PLoS One. 2016; 11(8):e0161782). However, a reduction in OA severity in a mild-to-severe model of OA over the course of 10 weeks with our current treatment regimen was not detected. In the bilateral ACLT model of OA, rats are not able to compensate during mobility with the contralateral control leg. The constant use of both joints may be increasing the inflammatory response to a level that our treatment dose or injection frequency with 24R,25 was not able to overcome. One limitation of this study is that we did not label 24R,25 and track the length of time it persists in the joint. Without being bound by theory, it may be that 24R,25 is cleared from the joint more quickly and that an increased injection frequency is needed to achieve chondroprotective effects.

### Conclusion

We successfully produced a mid-to-severe model of OA.

### Example 2

Cross-Talk Between miRs, TNF-α, and WNT Signaling

### Overview and Rationale

Packaging of microRNA in matrix vesicles was examined in the growth plate, wherein a set of 8-15 microRNAs were analyzed as they were uniquely expressed either in the matrix vesicles of the less mature costochondral resting zone chondrocytes or in the more mature costochondral growth zone chondrocytes. In order to determine the function of these specific microRNAs, these chondrocytes were transfected with different microRNAs and preliminary testing of the resulting DNA, proliferation, and glycosaminoglycans content was evaluated. One microRNA in particular, miR-122, (SEQ ID NO:1) was able to cause an increase in proliferation in both the resting zone and growth zone chondrocytes. miR-122 was selectively packaged in the matrix vesicles of these chondrocytes. We theorized this microRNA might be responsible for maintaining the pool of resting zone chondrocytes. Additionally, we hypothesized that if we encourage the damaged and dying chondrocytes in OA to proliferate, then we would have a means of repairing and maintaining the ECM. This would mitigate OA progression, or possibly prevent it altogether. We chose miR-451 as a control microRNA, as it was found to be uniquely packaged in the growth zone chondrocytes, and caused a decrease in total DNA and proliferation, indicating it may be encouraging terminal differentiation.

The following Examples with these microRNAs established their functions in articular chondrocytes in the presence and absence of the inflammatory cytokine IL-1β. Since IL-1β signaling is a major contributor of OA, a well-established model of creating an osteoarthritic phenotype in vitro is to stimulate cell cultures with this cytokine. This study first established that both these microRNAs were elevated in OA in the bilateral ACLT model. Transfection with miR-122 stimulated proliferation and was able to prevent the IL-1β-stimulated increase in catabolic matrix metalloproteinase-13 (MMP-13), inflammatory prostaglandin E2 (PGE2), and increased total DNA. The anti-inflammatory properties of miR-122 transfection prevented IL-1β-stimulated increase in other inflammatory mediators, cytokines, chemokines, and growth factors including IL-1α, IL-2, IL-4, IL-6, GM-CSF, MIP-1A, RANTES, and VEGF.

The miR-451 miR (SEQ ID NO:10) was originally chosen as a control miR but it had surprising and very specific functions. Transfection with miR-451 in the presence of IL-1β-stimulation led to exacerbated production in MMP-13 and PGE2, even more so than levels with IL-1β-stimulation alone. Interestingly, this microRNA did not encourage these responses in the absence of IL-1β. Further exploration of this indicated that miR-451 also increased the inflammatory mediators, cytokines, chemokines, IL-1α, IL-2, IL-4, IL-6, GM-CSF, and MIP-1A, while it decreased the growth factor VEGF. Without being bound by theory, these data may indicate that miR-451 targets an anti-inflammatory molecule, and thus, in the presence of inflammation, exacerbates synergistic increase of the downstream molecules. A series of transfections and inhibition experiments with these microRNAs confirmed that these changes were due to the microRNAs themselves. This Example demonstrates that miR-122 had a chondro-protective role in the presence of inflammatory signaling molecule IL-1β while miR-451 chondro-destructive role, causing an exacerbated inflammatory response in the presence of IL-1β. This and additional Examples further demonstrate the usefulness of administration or increased expression of miR-122 and/or the inhibition of miR-451 as therapeutic options for the treatment of OA.

IL-1β is one of the main drivers in the chronic dysregulated signaling present in OA, but it is not the only one. The crosstalk of IL-1β, TNF-α, and WNT/β-catenin signaling pathways, as well as the overwhelming evidence of their roles in driving OA progression make them attractive options to explore the mechanism of action and modulation of microRNAs as OA therapeutics. The following Examples demonstrate the roles of miR-122 and miR-451 through the TNF-α and WNT/β-catenin signaling pathways. WNT/β-catenin agonist LiCl had a protective effect against both MMP-13 and PGE2 in IL-1β stimulated cultures and against PGE2 in TNF-α stimulated cultures in rArCs. WNT/β-catenin antagonists XAV and PKF exacerbated the IL-1β-stimulated increases in MMP-13 and PGE2. However, XAV only exacerbated the TNF-α-stimulated increase in PGE2 and not MMP-13. miR-122 protected against both IL-1β and TNF-α stimulated increases in MMP-13 and PGE2 production, potentially by targeting WNT/β-catenin; miR-451 did not signal through the TNF-α pathway. Interestingly, miR-451 did not exacerbate inflammatory signaling through a TNF-α affected means, indicating its mechanism of action is targeting a specific mediator in the IL-1β signaling pathway other than TNF-α.

Interleukin-1β (IL-1β), tumor necrosis factor-α (TNF-α), and WNT/β-catenin signaling cause dysregulation of rat primary articular chondrocytes (rArCs), resulting in degradation of cartilage extracellular matrix and chondrocyte death in vitro and help drive osteoarthritis (OA) progression in vivo. microRNA (miR) miR-122 represses these effects in vitro whereas miR-451 exacerbates IL-1β-stimulated matrix metalloproteinase-13 (MMP-13) and prostaglandin E2 (PGE2) production. The goals of this study were to evaluate crosstalk between these signaling pathways and determine if miR-122 and miR-451 exerting their protective or destructive effects through one or more aspects of these pathways.

rArCs were treated with WNT agonist lithium chloride (LiCl), WNT antagonist XAV-939 (XAV), or PKF-118-310 (PKF) with and without IL-1β or TNF-α stimulation. Additionally, cultures were transfected with miR-122, miR-451, miR-122-inhibitor, or miR-451-inhibitor and treated with TNF-α. Total DNA, MMP-13, and PGE2 were measured.

On a microscale, a phenotypic shift of the normally quiescent articular chondrocytes leads to the aberrant expression of pro-inflammatory and catabolic pathways, which are hypothesized to contribute to OA progression. Of note, interleukin-1β (IL-1β) and tumor necrosis factor-α (TNF-α) are key players disrupting cartilage homeostasis and driving inflammatory pathway activation. Both cytokines are elevated in the synovial fluid of OA patients, and can remain chronically elevated more than one year after an injury to the anterior cruciate ligament (ACL). These pathways become dysregulated and form a positive feedback loop, increasing their own production and that of other inflammatory cytokines such as prostaglandin E2 (PGE₂); matrix-degrading proteases, including a disintegrin and metalloproteinase with thrombospondin motifs-4 and 5 (ADAMTS-4, ADAMTS-4) and matrix metalloproteinase-1, 3 and 13 (MMP-1, MMP-3, MMP-13); and, reactive oxygen species like nitric oxide (NO).

As both IL-1β and TNF-α signaling pathways converge on the inflammatory NF-κB signaling pathway, it is not surprising they elicit increases in many of the same inflammatory and catabolic markers. Their involvement in OA progression has been well documented. Interestingly, TNF-α stimulation has been positively linked to an increase in canonical WNT-3a and WNT-7a and IL-1β stimulation positively increases β-catenin, which also signals through the NF-κB signaling pathway during inflammation. Mounting evidence has suggested the WNT/β-catenin signaling pathway plays a pivotal role in OA progression as well. Both mediators and downstream targets of WNT/β-catenin signaling are elevated in OA. Both canonical and non-canonical WNT signaling is linked to the expression of MMP-3, MMP-3, MMP-13, MMP-14, ADAMTS-4, and ADAMTS-5, and induces cartilage degradation. Additionally, inhibiting WNT/β-catenin using PKF115-584, and PKF118-310 block IL-1β and TNF-α induced cartilage degradation, and inhibition of WNT/β-catenin using XAV-939 in a destabilization of the medial meniscus model of OA in mice reduced cartilage degradation.

Increasing evidence suggests that disease pathologies can be enhanced or inhibited through microRNA (miR) regulation, and specific microRNAs such as miR-17-5p, miR-21-5p, miR-101, miR-451a, are elevated in OA (see Scott, supra; Wang et al. Rheumatol (UK). 2019; 58(8):1485-97; and Li et al. Exp Ther Med. 2017; 1789-94). Specifically, recent findings found that miR-122-5p prevents the associated increase in MMP-13, PGE₂, and other cytokines implicated in OA in primary rat articular chondrocytes upon stimulation with IL-1β. Conversely, miR-451-5p exacerbated the increase of MMP-13, PGE₂, and numerous other cytokines implicated in OA, but only in the presence of IL-1β. These microRNAs were found elevated 10 weeks following an anterior cruciate ligament transection model of OA in Sprague Dawley rats, suggesting they are playing a role in the disease pathology. Prior to these studies, these microRNAs have only been examined after IL-1β stimulation, and if or how they may be cross-talking with the TNF-α and WNT/β-catenin signaling pathways has yet to be elucidated.

The crosstalk of IL-1β, TNF-α, and WNT/β-catenin signaling pathways, as well as the overwhelming evidence of their roles in driving OA progression make them attractive options to explore the mechanism of action of miR-122 and miR-451. The goal of the present study was to evaluate how miR-122 and miR-451 may be cross-talking with TNF-α, and WNT/β-catenin pathways to elicit their anti-inflammatory or pro-inflammatory actions in the presence of inflammatory stimuli.

### Materials and Methods

### Articular Chondrocyte Isolation

Rat articular chondrocytes (rArCs) were isolated using a previously established protocol (see Scott, supra). Briefly, rArCs were isolated from femoral condyles and tibial plateaus from 100-125 g male Sprague Dawley rats. Cartilage was sterilely sliced from the articular surfaces using a surgical blade and placed in Dulbecco's modification of Eagle's medium with 1 g/L glucose, 50 U/mL penicillin and streptomycin (pen-strep) (DMEM). After removing DMEM, cartilage was incubated with 0.05% trypsin (20 mins, 37° C.), rinsed with DMEM (50 U/mL pen-strep, 20 mins, 37° C.), and then digested with sterile-filtered 0.03% collagenase type II solution (Worthington Biochemical, Lakewood, NJ) in Hank's balanced salt solution (16 hrs, 37° C.). Digestion was quenched with an equal volume of DMEM containing 10% fetal bovine serum (FBS) and 50 U/mL pen-strep, strained through a 40 μm strainer (Greiner Bio-one, North Carolina), spun down at 500×g for 10 mins and plated at a seeding density of 15,000 cells/cm². These cells are at passage 0. All experiments used cells at passage 1.

### Cell Culture

rArCs were plated at a seeding density of 15,000 cells/cm² and fed with DMEM supplemented with 10% FBS and 50 U/mL pen-strep. Media were changed 24 hours after plating and then every 48 hours. After treatments described below, conditioned media were collected, centrifuged at 1,000×g for 10 mins to remove cellular debris, aliquoted, and stored at −80° C. until assayed. Cell monolayers were rinsed twice with 1 mL of 1×PBS, lysed in 500 μL of 0.05% Triton X-100, and frozen for later DNA analysis. MMP-13 (ANASpec, Fremont, CA) and PGE2 (R&D Systems) were measured in the conditioned media by a microplate reader at ex/em 490 nm/520 nm, and 450 nm absorbance, respectively, in accordance with the manufacturer's directions. Data were normalized to the DNA content of the cell monolayer. Cell layers were thawed, and mechanically lysed by ultrasonication (40V, 10s/well) (VCX-120; Vibra-cell, Newtown, CT) and DNA was measured using QuantiFluor dsDNE system kit (Promega, Madson, WI) and quantified on a fluorescence plate reader at ex/em 504 nm/531 nm.

### Mimic and Inhibitor Transfections

Cell transfection was performed using a previously established protocol (see Scott, supra). Briefly, at 60% confluence, rArCs were transfected for 24 hours with 14.5 nM of mirVana miRNA mimic rno-miR-122-5p (SEQ ID NO:2) or rno-miR-451-5p (SEQ ID NO:4) (Thermo Fisher Scientific) using 0.2% lipofectamine RNAimax (Lipo, Life Technologies, Carlsbad, CA) in antibiotic-free DMEM supplemented with 10% FBS. Lipo groups served as a negative control and contained 0.2% Lipo. LNA Inhibitor transfection was performed with rno-miR-122-5p miRCURY or rno-miR-451-5p miRCURY LNA miRNA-inhibitors (Qiagen). Cells were transfected at 60% confluence using 0.2% Lipo and 30 nM inhibitor for 24 hours. Following mimic or inhibitor transfection, fresh media with or without 10 ng/mL TNF-α (Peprotech, Rocky Hill, NJ) were added for an additional 24 hours and then harvested as described above.

### WNT Agonist Experiments

The canonical WNT agonist lithium chloride (LiCl) was obtained from Millipore Sigma (Burlington, MA). Dosing concentrations were determined from a previously published protocol (see Minashima et al. Arthritis Rheumatol. 2014; 66(5):1228-36). rArCs were treated at 80% confluency with 0, 5 or 10 mM LiCl for 24 hours. Following treatment, media were replaced with fresh media with or without 10 ng/mL of IL-1β (Peprotech) or 10 ng/mL TNF-α for 24 hrs and then harvested as described above.

### WNT Inhibitor Experiments

XAV-939 (XAV) and PKF-118-310 (PKF) were obtained from Millipore Sigma (Burlington, MA). XAV is a tankyrase 1 and 2 inhibitor that works by stabilizing AXIN2 in the cytosol, while PKF selectively inhibits β-catenin from complexing with TCF-4 in the nucleus. Concentrations for XAV and PKF were chosen based on previously published studies (see Dittfeld et al. PLoS One. 2018; 13(12):e0208774; Huang et al. Nature. 2009; 461(7264):614-20; and Ma et al. J Clin Invest. 2016; 126(5):1745-58). rArCs were treated at 80% confluence with 0 μM, 0.5 μM, or 1 μM of XAV or 0 μM, 0.1 μM, 0.5 μM, or 1 μM of PKF, or with or without 10 ng/mL of IL-1β or ng/mL TNF-α for 24 hrs and then harvested as described above. IL-1β and TNF-α were added to culture media at the same time as WNT inhibitors to assess the interactions of their pathways.

### Gene Expression

To determine if TNF-α stimulation increases miR-122 or miR-451 expression, rArCs were grown to 80% confluence and treated with or without 10 ng/mL TNF-α for 12 hrs. RNA was isolated from cells using the Qiagen RNeasy mini kit, quantified (Tank3 Micro-Volume Plate, Biotek, Winooski, VT), and cDNA was generated using the miScript II RT Kit (Qiagen). Quantitative PCR was performed using miScript SYBR® Green PCR Kit (Qiagen) with miR specific primers and normalized to U6 small nuclear 1 (RNU-6, Mysticq® microRNA qPCR Control Primer, Sigma-Aldrich, St. Louis, MO).

### Statistical Analysis

Data are from single experiments and are represented as mean±standard error (n=6 independent cultures per variable). Each experiment was repeated independently a minimum of two times to ensure the validity of the results. A one-way analysis of variance was performed followed by a two-tailed Tukey correction using an alpha equal to 0.05 as denoted by letters. All statistical analyses were performed using GraphPad Prism version 5.04 or JMP Pro 14.

### Results

Dose-Dependent Treatment with TNF-α

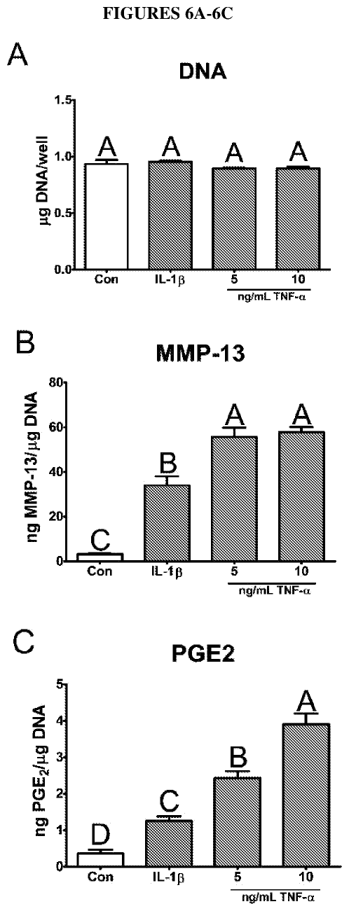

There were no differences in DNA between the control and treatment groups, shown in FIG. 6A. An increase was seen in MMP-13 production after IL-1β treatment, as shown in FIG. 6B. Treatment with both 5 and 10 ng/mL of TNF-α caused a significant increase in MMP-13 production compared to both IL-1β treated and control groups. These results were confirmed when evaluating PGE2 production. Similarly, IL-1β, as well as both 5 and 10 ng/mL of TNF-α treatment significantly increased PGE2 production compared to control levels, with the greatest increase in the 10 ng/mL TNF-α treated group, shown in FIG. 6C. A concentration of 10 ng/mL TNF-α was used for later experiments.

miR-122 Crosstalk with the TNF-α Pathway

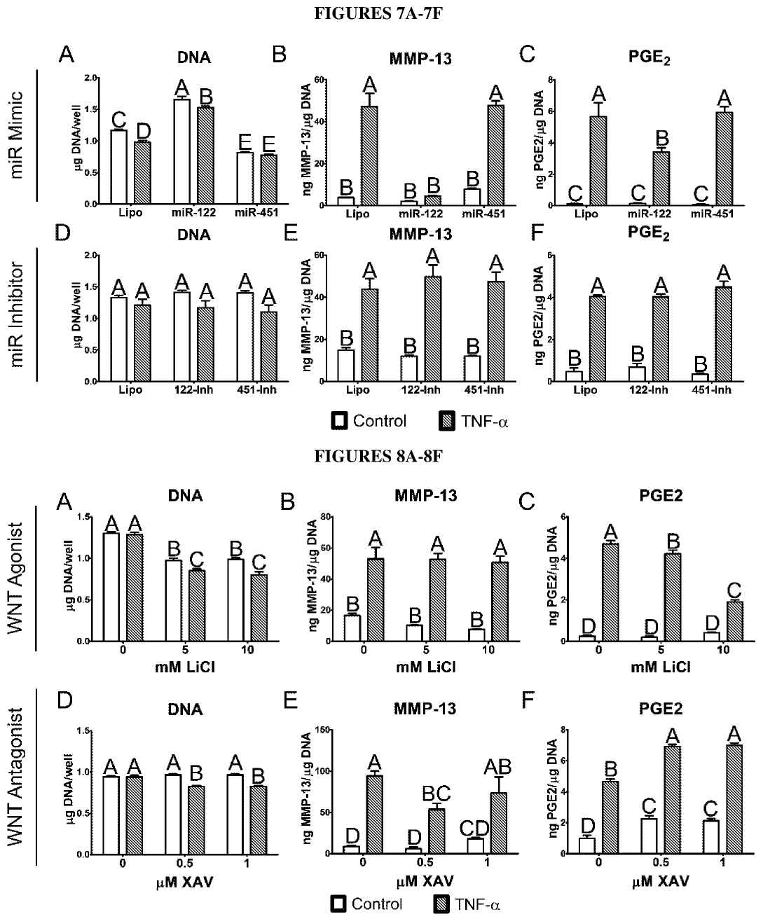

Previous data showed that transfection with miR-122 was able to inhibit the IL-1β-stimulated increase in MMP-13 and PGE2 while transfection with miR-451 exacerbated the production of these two molecules (see Scott, supra). We wanted to evaluate these microRNAs mechanism of action in the TNF-α pathway. miR-122 transfection increased total DNA while miR-451 transfection reduced total DNA compared to all groups as shown in FIG. 7A, confirming previous findings. Treatment with TNF-α modestly decreased total DNA in the lipo control and miR-122 treatment groups but did not change total DNA in miR-451 transfected cells (also shown in FIG. 7A). TNF-α stimulation increased both MMP-13 and PGE2 compared to its respective controls, shown in FIGS. 7B and 7C. Transfection with miR-122 prevented the TNF-α-stimulated increase in MMP-13 production; interestingly while transfection with miR-122 did decrease PGE2 production compared to the TNF-α stimulated control, it did not rescue it completely back to non-TNF-α stimulated control levels. Conversely, transfection with miR-451, with or without TNF-α stimulation, did not change MMP-13 or PGE2 levels, shown in FIGS. 7C and 7D.

Next, we evaluated inhibiting miR-122 and miR-451 with and without TNF-α stimulation. There were no changes in total DNA in any of the treatment groups, shown in FIG. 7D. TNF-α stimulation increased both MMP-13 and PGE2 compared to its respective controls, shown in FIGS. 7E and 7F. Inhibition of both miR-122 and miR-451 did not change MMP-13 or PGE2 production.

WNT/β-Catenin Cross-Talk with TNF-α Pathway

In order to evaluate the crosstalk between the TNF-α and WNT/β-catenin pathways, cultures were treated with the WNT/β-catenin agonist, LiCl, or an antagonist, XAV, with or without TNF-α treatment, shown in FIGS. 8A-8F. Treatment with TNF-α did not change total DNA in the control group. Both the 5 mM and 10 mM treatment with LiCl decreased total DNA to similar levels; treatment with TNF-α in those groups marginally decreased this (FIG. 8A). The expected increases in MMP-13 and PGE2 production were seen after stimulation with TNF-α in the control groups (FIG. 8B, 8C, 8E, 8F). There were no differences in MMP-13 production in either the 5 mM or 10 mM LiCl group with or without TNF-α treatment compared to their respective controls. Treatment with 5 mM or 10 mM LiCl alone did not change PGE2 production, however, a dose-dependent decrease was seen in the presence of TNF-α (FIG. 8C). Treatment with the WNT inhibitor XAV caused a decrease in total DNA in the presence of TNF-α stimulation (FIG. 8D). Treatment with XAV alone did not change MMP-13 production compared to control levels. 0.5 μM XAV decreased the TNF-α-stimulated increase in MMP-13, but this effect was not present in the 1 μM XAV+TNF-α group (FIG. 8E). Conversely, XAV had a greater stimulatory effect on PGE2 production on its own and caused increases in both the 0.5 μM and 1 μM XAV groups (FIG. 8F). This stimulatory effect acted synergistically in the presence of TNF-α stimulation (FIG. 8F).

WNT/β-Catenin Crosstalk with IL-1β Pathway

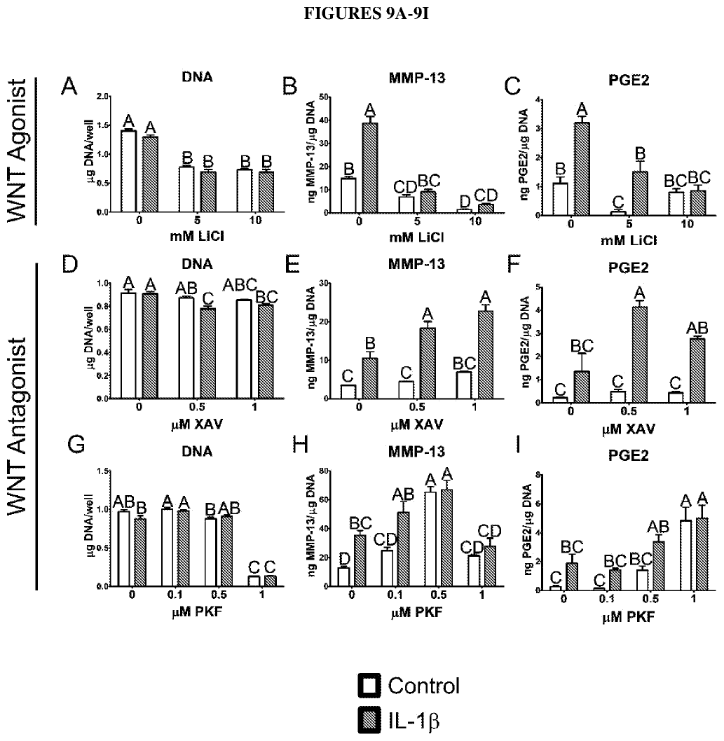

In order to evaluate how the crosstalk between the IL-1β and WNT/β-catenin pathways, cultures were treated with the WNT/β-catenin agonist, LiCl, or with two different antagonists, XAV or PKF with or without IL-1β treatment as shown in FIGS. 9A-9I. Both the 5 mM and 10 mM LiCl treatment decreased total DNA as previously seen (FIG. 9A). Stimulation with IL-1β increased both MMP-13 and PGE2 as expected (FIG. 9B, 9C). Increasing concentrations of LiCl resulted in a decrease in MMP-13 production both in the presence and absence of IL-1β (FIG. 9B). A similar trend was seen in PGE2 production (FIG. 9C).

There was a slight decrease in total DNA with treatment of both XAV and IL-1β, however, this was not seen in groups treated with only XAV (FIG. 9D). There were no differences in MMP-13 or PGE2 production with increasing concentrations of XAV alone; however, XAV treatment with IL-1β-stimulation showed synergist increases in both MMP-13 and PGE2 (FIG. 9E, 9F). Concentrations of 1 μM PKF, but not 0.1 μM or 0.5 μM PKF, drastically decreased total DNA (FIG. 9G). There was a dose-dependent increase in MMP-13 production with increasing concentrations of PKF alone with the exception of the 1 μM group (FIG. 9H). An expected increase was seen in MMP-13 with IL-1β-stimulation in the 0.1 μM PKF, but this was not present in the 0.5 μM or 1 μM PKF IL-1β-stimulation groups (FIG. 9H). There was no difference in the 1 μM PKF groups both with and without IL-1β-stimulation compared to control groups (FIG. 9H). A dose-dependent increase in PGE2 production was seen with increasing concentrations of PKF (FIG. 9I), with the largest increase in the 1 μM PKF groups both with and without IL-1β-stimulation. The IL-1β-stimulation increase in PGE2 was not seen in these groups.

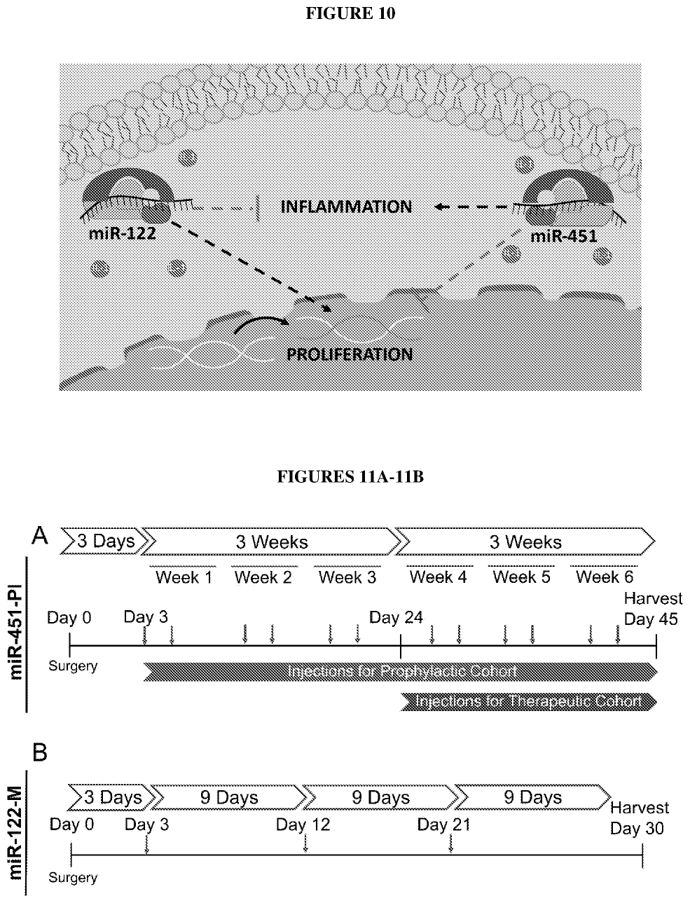

Without being bound by theory, FIG. 10 illustrates a proposed mechanism of miR-122 and miR-451 in rat articular chondrocytes. This diagram shows how miR-122 appears to signal through both TNF-α and IL-1β signaling pathways, while miR-451 only acts through IL-1β. Stimulation with TNF-α or IL-1β causes increases in downstream inflammatory mediators as well as MMP-13 and PGE2. Transfection with miR-122 prevents the associated increase in MMP-13 and to a lesser extent, in PGE2 with TNF-α stimulation. miR-122 transfection inhibits both the increase in MMP-13, and PGE2 to the same extent with IL-1β stimulation. Transfection with miR-451 in the presence of IL-1β exacerbates MMP-13 and PGE2 production, however miR-451 does not affect the TNF-α signaling pathway.

### Discussion

IL-1β, TNF-α, and WNT/β-catenin signaling are dysregulated in OA and research has indicated they drive OA progression, as shown in FIG. 10. Numerous studies examine how these pathways each individually contribute to OA. Inhibition of TNF-α decreases OA severity in both spontaneously and surgically induced OA models in mice and in experimentally induced OA in rabbits. Additionally, TNF-α inhibition promotes the repair of osteochondral lesions and has chondroprotective effects in vivo. However, Zwerina and colleagues found that articular cartilage changes caused by overexpression of TNF-α are not fully blocked by either TNF-α or IL-1 inhibition alone, but the combined blockade of both TNF-α and IL-1 lead to almost complete remission of the disease (supra). These observations suggest that multiple aberrant pathways influence OA progression, as illustrated in FIG. 10. Our data support this hypothesis and indicate that there is crosstalk between them.

We took advantage of the differential regulation of inflammatory processes in rat articular chondrocytes by two microRNA found in OA cartilage, miR-122 and miR-451, to examine whether crosstalk occurs. We found that miR-122 transfection was able to prevent the associated TNF-α stimulated increase in MMP-13 and, to a lesser extent, in PGE2. Taken together with our previous data (Scott, supra), miR-122 is influencing genes that crosstalk with both the IL-1β and TNF-α signaling pathways. In contrast, miR-451 appears to exacerbate the MMP-13 and PGE2 production only in IL-1β stimulated cultures and does not appear to crosstalk or overlap with the TNF-α stimulated increase in these markers. Interestingly, no change in MMP-13 and PGE2 production was detected when these miR-122 or miR-451 were inhibited. This may be due to low endogenous expression levels of miR-122 in our culture system. Alternatively, this may be because miR-122 controls downstream targets of TNF-α but does not directly control TNF-α so we do not see any effects of inhibiting miR-122.

WNT/β-catenin signaling plays a predominant role in OA; however, this pathway remains extremely complex. There is a delicate balance of WNT signaling needed to maintain cartilage homeostasis. Elevated levels of β-catenin, WNT-5B, WNT-7B, WNT-10B, WNT-11, and WNT-16 have been found in OA (Zwerina, supra; Wang et al. Cell Commun Signal. 2019 August; 17(1):97; Hwang et al. J Biol Chem. 2004; 279(25):26597-604. Both repression and prolonged activation of β-catenin cause articular cartilage degradation. Preventing β-catenin degradation (therefore, more β-catenin signaling) resulted in an OA-like phenotype with articular cartilage loss and osteophyte formation (see Zhou et al. Curr Rheumatol Rep. 2017 September; 19(9):53). Conversely, inhibiting (3-catenin also caused articular cartilage destruction (see Wang, supra).

Activation of β-catenin in mature chondrocytes has been linked to increased apoptosis (see Ning et al. Int Orthop. 2012; 36(3):655-64). We saw a decrease in total DNA with both the 5 mM and 10 mM LiCl treatment indicating there may have been an increase in apoptotic events with our treatment. There are conflicting reports of LiCl treatment and resulting MMP-13 expression in the literature. We saw a decrease in MMP-13 and PGE2 protein production in LiCl-treated cultures with IL-1β-stimulation, and a decrease in PGE2 production in the LiCl and TNF-α-stimulated cultures, but not in MMP-13 production. This may indicate that TNF-α stimulation causes an increase in MMP-13 through a slightly different signaling mechanism that the protective effects of LiCl were not able to mitigate. Similar to our findings, multiple studies have examined LiCl treatment in human and bovine chondrocytes and found a decrease in both MMP-13 gene expression and protein production in IL-1β-stimulated cultures. These results contradict another study that shows that LiCl treatment with IL-1β-stimulation increases MMP-13 expression in rabbit chondrocytes, while others found an increased MMP-13 expression with LiCl treatment alone. These conflicting results may be explained by the fact that both too little and too much (3-catenin signaling can cause OA-phenotypical changes.

When we used the cytosolic β-catenin inhibitor XAV in the TNF-α stimulation culture we saw an increase in PGE2 production, which agrees with our LiCl data. The effect on MMP-13 production was minimal, similar to our findings in the LiCl treated cultures. A similar trend was seen when we inhibited with both the cytosolic and nuclear inhibitor XAV or PKF in the IL-1β-stimulated cultures. Both MMP-13 and PGE2 production was exacerbated in groups treated with both the inhibitor and IL-1β compared to IL-1β alone. These data suggest that there is crosstalk between TNF-α, IL-1β, and WNT/β-catenin signaling pathways and that targeting one pathway alone may not totally negate the inflammatory signaling found in OA.

Together, these data indicate complex communication between these pathways; LiCl has a protective effect against IL-1β stimulation, while it can only protect against PGE2 expression, and not MMP13, in TNF-α stimulated cultures. Interestingly, miR-122 appears to target FOXO3 which modulates the WNT/β-catenin signaling pathway, which may explain miR-122's protective role in IL-1β and TNF-α stimulated cultures. miR-451 transfection exacerbates the production of MMP-13 and PGE2 in the presence of IL-1β stimulation but has no inflammatory signaling in the absence of IL-1β. Furthermore, miR-451 had no effect in TNF-α stimulated cultures, indicating it is targeting a specific branch or inhibitor in the IL-1β signaling pathway.

Identification of the specific signaling mechanism that these two microRNAs are targeting will explain their protective and destructive effects in articular cartilage. Additionally, examining treatment with miR-122 and inhibition of miR-451 in an OA model will demonstrate their chondroprotective role in this complex disease pathology.

### Conclusion

LiCl has protective effects against the IL-1β-stimulated increase in MMP-13 and PGE2, and in the TNF-α stimulated increase in PGE2, but not MMP-13. MiR-122 is able to protect against the associated increase in MMP-13 and PGE2 in both TNF-α and IL-1β-stimulated cultures. MiR-451 does not appear to exacerbate inflammatory signaling through a TNF-α affected means.

### Example 3

In Vivo Treatment with miR-122 Inhibitor or miR-122 Mimic in a Bilateral ACLT Model of OA

Two microRNA found in articular cartilage, miR-451 and miR-122, have differential effects in vitro when chondrocytes are treated with IL1β: miR-451 exacerbates IL1β effects whereas miR-122 stimulates chondrocyte proliferation. We used a rat ACLT model to examine the prophylactic and therapeutic potential of inhibiting miR-451 as well as the prophylactic potential of miR-122 mimic by evaluating treatment at the onset of joint trauma and treatment after OA has developed. Two animal cohorts were used to evaluate miR-451-power inhibitor (451-PI). The prophylactic cohort received twice-weekly intra-articular injections of either 451-PI or a negative control (NC-PI) beginning on post-surgical day 3. In the therapeutic cohort, OA was allowed to develop for 24 days before beginning injections. All rats were killed on day 45. In another study, either miR-122 mimic (122-M) or a negative control (NC-M) were delivered via intra-articular injections on days 3, 12, and 21; rats were killed at day 30 (3 total injections). Micro-CT, histomorphometric analyses, OARSI scoring, and muscle force testing were performed on samples.

Differing microRNA (miR) profiles have been found in diseased tissue compared to their healthy counterparts, indicating miRs may be influencing disease development. miR-9, miR-27, miR-34a, miR-139, miR-140, and miR-146a are elevated in OA. Their expression is increased with IL-1β stimulation in vitro and they control cellular processes such as apoptosis and matrix metalloproteinase-13 (MMP-13) mediated degradation of articular cartilage extracellular matrix (ECM).

Previous RNAseq analysis of rat costochondral cartilage growth plate chondrocyte cultures revealed miR profiles vary with the developmental zone from which the cells were isolated. miRs are packaged in matrix vesicles (MVs) produced by their parent cells and incorporated into their extracellular matrix. As discussed supra, miR-122-5p and miR-451a were found specifically enriched in growth plate MVs, indicating they may play a role in regulating chondrocyte maturation and homeostasis. Further analysis of these microRNAs in vitro using primary rat articular chondrocytes demonstrated that miR-122 prevented IL-1β-induced increases in catabolic MMP-13 and inflammatory prostaglandin E2 (PGE2), whereas miR-451 exacerbated this response. Additionally, elevated levels of miR-451 expression were found in OA knees of Sprague Dawley rats 10 weeks following anterior cruciate ligament transection (ACLT) compared to sham knees. These data suggest that miR-451 may be exacerbating OA progression, while miR-122 may be able to decrease OA progression. The goal of this Example was two-fold: 1) to assess the prophylactic and therapeutic potential of inhibiting miR-451 in an established rat post-traumatic ACLT model of OA by evaluating treatment at the onset of joint trauma and treatment after OA has developed; and 2) to evaluate 122 mimic in preventing OA.

### Materials and Methods

### Rat Anterior Cruciate Ligament Transection (ACLT) Model of OA

A total of 31 skeletally mature male Sprague-Dawley rats (Envigo, Indianapolis, IN), 9-10 weeks of age, weighing 250-300 g at the study onset, were used for these experiments. All animal procedures were performed according to the National Research Council's Guide for the Care and Use of Laboratory Animals and approved by Virginia Commonwealth University's Institutional Animal Care and Use Committee. Animals were fed ad libitum. A 30% effect size, 20% variance, and a power of 80%, a two-tail analysis indicated an n=8 per group was necessary to yield statistical significance based on a previous study (Scott et al. Osteoarthr Cartil. 2020; 29:113-23).

Two in vivo experiments were performed. The first included twenty animals. Each treatment group had n=8 plus 1 extra animal in case of morbidity or mortality. Nine of the rats were used to examine the prophylactic effect of 451-PI. Another nine animals were used to examine the therapeutic effect. Two animals were used as non-operated controls. A total of eleven rats was used to assess the effectiveness of 122-mimic at preventing the development of OA. Eight animals were used for the treatments, as described below. Based on the muscle physiology data generated in the first experiment, we increased the number of control rats to 3.

Treatment with 451-PI to Prevent OA Progression

The miR-451-PI study initially included twenty 9-10-week-old male Sprague Dawley rats. One rat died during surgery resulting in nineteen total animals, of which seventeen were sedated 2-4% isoflurane anesthesia in 400 mL/min 02 to effect. Bilateral anterior cruciate ligament transections (ACLT, animals=18) were performed by medially translocating the patellar tendon in order to visualize and transect the ACL to create a severe model of OA.

The rats were split into a prophylactic and a therapeutic cohort. In both cohorts, the left hind legs received injections of rno-miR-451a (custom miRCURY LNA power inhibitor, 451-PI). Right hind legs received the negative control molecule (NC-PI). Both 451-PI and NC-PI were modified for in vivo use (PS, HPLC+Na+salt exchange) (Qiagen, Hilden, Germany). In order to evaluate the preventative potential of miR-451-inhibition, three days following surgery, the prophylactic cohort (n=9 rats, 9 legs/treatment group) received 50 μL intra-articular injections of 100 nM of 451-PI (left legs) and NC-PI (right legs) twice a week (Error! Reference source not found.A). Two animals were left untouched to serve as aged-matched controls and all four hind legs were used as controls (normal). The same controls were used for both cohorts in this study.

In order to evaluate the therapeutic potential of 451-PI, a therapeutic cohort was included that allowed OA to develop for 3 weeks before administering treatments at 3 weeks post-surgery for a total duration of 3 weeks. The therapeutic cohort was initially n=9 rats (9 legs/treatment group). However, one animal died resulting in n=8 legs/treatment group). All animals received the same dose and injection regimen as the prophylactic cohort, but the treatment did not begin until day 24, rather than 3 days following surgery (FIG. 11A). Six weeks following the onset of treatment (post-surgery day 45), in vivo muscle force testing was conducted on the tibialis anterior (TA) while animals were sedated. Following muscle testing, animals were euthanized according to IACUC standards.

Treatment with 122-Mimic to Prevent OA

Eleven 9-10-week-old male Sprague Dawley rats were used in this study. Eight animals underwent bilateral ACLT, as described above. In order to evaluate the prophylactic potential of 122-M, three days following surgery, animals received 50 μL intra-articular injections of 2 mg/mL of either rno-miR-122 mimic 2.0 (HPLC purified, Life Technologies, Carlsbad, CA), or rno-negative control #1. The injection leg (right vs left hind leg) was randomized for each animal (animals=8, n=8 legs/treatment group). Injection dose was modified from a previously published protocol (Dai et al. Mol Ther. 2015 August123(8):1331-40). Mimics were conjugated to the delivery vehicle invivofectamine 3.0 reagent (Thermo Fisher Scientific, Waltham, MA) according to the manufacture's protocol. The injections were given on days 3, 12 and 21, for a total of three injections (FIG. 11B). Three animals were left untouched to serve as aged-matched controls and all six hind legs were used as controls (normal). In vivo muscle force testing was conducted on the tibialis anterior (TA) while animals were sedated. Following muscle testing, animals were euthanized according to IACUC standards 30 days after surgery.

### MicroCT

Samples were fixed in 10% buffered formalin for 3 days. Scans were performed using a resolution of 2240×2240 (image pixel size of 7.91 μm), 45 kVp, 177 μA, 1300 ms exposure and rotation step size of 0.35 degrees. All scans were performed using the same settings. Subchondral bone measurements were performed by drawing a region of interest (ROI) tightly around the subchondral bone plate. For areas where the subchondral bone was remodeled and no longer present, the ROI was drawn as a projection of where the bone would be. The bone volume (BV) and total volume (TV) of the ROI were measured and a B V/TV percent was reported for the medial condyle (med BV/TV), lateral condyle (Lat), and added together (total). MicroCT for the 451-PI study resulted in an n=4 for controls, n=9/treatment for the prophylactic cohort and an n=7/treatment for the therapeutic cohort (one animal's scan did not reconstruct properly due to corrupt files and one animal died during surgery). MicroCT for the 122-M study resulted in an n=6 for the control and n=8/treatment.

### Histology

Samples were decalcified in 40 mL of 14% EDTA tetrasodium salt dehydrate (Millipore Sigma, Burlington, MA) containing 1.8% glacial acetic acid (Spectrum Chemical, Gardena, CA), pH 7.4-7.6, for 6-8 weeks. Solutions were changed twice weekly. Samples were rinsed in running deionized water for 15 min, dehydrated in a series of 70%, 95% and 100% ethanol and xylene washes, and embedded with Richard-Allan Scientific Histoplast Paraffin (Thermo Fisher Scientific). Sections (5 μm) were collected using a manual microtome (Shandon Finesse 325, Thermo Fisher Scientific) and stained using Masson's trichrome. Samples were imaged using Zen 2012 Blue Edition software with an AxioCam MRc5 camera and Axio Observer Z.1 microscope (Carl Zeiss Microscopy, Oberkochen, Germany).

### Histomorphometrics

Articular cartilage and fibrotic area of medial femurs were measured using Zen 2012 Blue Edition software. The subchondral bone plate was measured and subdivided into healthy regions (normal cartilage above) and unhealthy regions (subchondral bone remodeling, osteophyte formation, and cartilage erosion/delamination). The percent healthy subchondral bone was reported as healthy subchondral bone divided by total subchondral bone*100. Histomorphometrics for the 451-PI prophylactic cohort study resulted in an n=4 for controls, n=9 for the NC-PI group and n=8 for the 451-PI group due to histological processing errors. Histomorphometrics for the therapeutic cohort resulted in an n=8/treatment. Histomorphometrics for the miR-122-M study resulted in an n=5 for the control, n=7 for the NC-M group and, n=6 for the 122-M group due to histological processing errors.

### OARSI Scoring

Performed as discussed in previous Examples.

### Muscle Force Testing

Prior to euthanasia, in vivo muscle force testing was conducted on the tibialis anterior muscles using the 1300A Whole Animal System (Aurora Scientific, Canada). Animals were sedated (2-4% isoflurane/400 mL/min O2) and placed in a supine position on the platform. Each leg was immobilized at the knee using a screw clamp and the paw was pressed firmly onto a pedal and secured with surgical tape. Two electrodes were inserted subcutaneously above the TA muscles to deliver electrical impulses for twitch and tetanic contractions. The foot flexes the pedal upon stimulation to produce a force-time curve. Twitch testing was performed 3 times using a 0.2 msec pulse width and the average of three tests was reported. Tetany testing was performed 3 times at 140 hz with a 500 m-sec stimulation duration until full muscle recruitment was reached followed by 120 s rest periods between stimulations to allow for muscle recovery. The average of three tests was reported. Muscle force testing was performed on all animals.

### Statistical Analysis

Data are represented as mean±standard error. The Grubbs' test was used to determine statistical outliers using an alpha=0.05. An asterisk indicates significance using a one-way analysis of variance followed by a two-tailed Tukey correction using an alpha=0.05. All statistical analyses were performed using GraphPad Prism version 5.04 or JMP Pro 14.

### Results

451-PI Decreased Evidence of OA when Used Prophylactically Following ACLT.

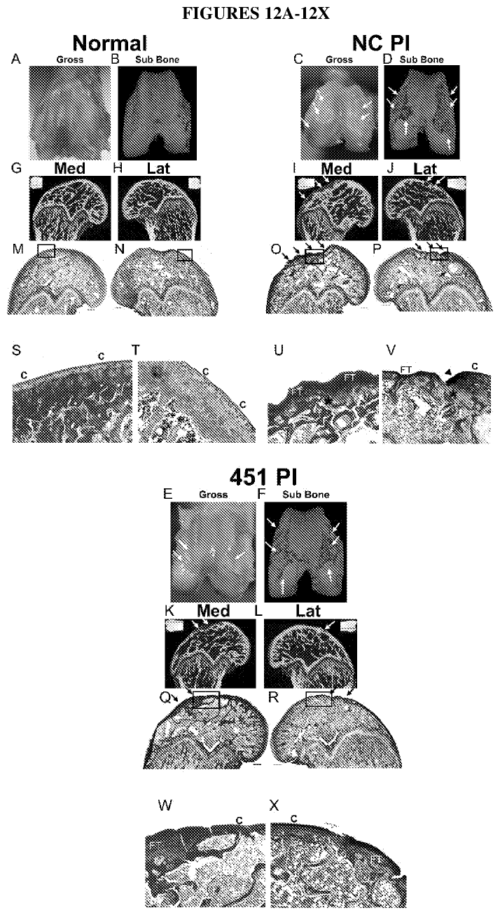

Analyses of OA following 451-PI treatment are shown in FIGS. 12A-12X. Gross images and 3D micro-CT reconstructions of femurs for the control (FIG. 12A, 12B), NC-PI (FIG. 12C, 12D) and 451-PI legs (FIG. 12E, 12F) showed evidence of OA. Cartilage lesions and subchondral bone erosion (white arrows) were present in the NC-PI and 451-PI limbs as compared to control limbs. This was confirmed in micro-CTs of the medial and lateral femurs; normal aged controls did not have irregularities in the subchondral bone (FIG. 12G, 12H), whereas subchondral bone erosion and remodeling could be seen (white arrows) in the NC-PI injected knees (FIG. 12I, 12J), and 451-PI injected knees (FIG. 12K, 12L). Most of the damage to the ACLT joints was to the medial aspect, although some cartilage erosion was also evident on the lateral aspect. Masson's trichrome stained medial and lateral femurs showed normal cartilage morphology in the controls (FIG. 12M, 12N), in contrast to the damaged articular cartilage (black arrows) in NC-PI (FIG. 12O, 12P), and 451-PI injected knees (FIG. 12Q, 12R).

At higher magnification, smooth articular cartilage surface (C) and healthy subchondral bone plates were seen in the controls (FIG. 12S, 12T). Higher magnification of NC-PI (FIG. 12U, 12V) and 451-PI (FIG. 12W, 12X) limbs revealed abnormal cartilage remodeling (black arrowheads) and damage to the subchondral bone plate. There were microfractures and irregularities in the subchondral bone plate (asterisks), with more damage present in the NC-PI limbs. Fibrotic tissue (FT) was present above the subchondral bone plate where normal articular cartilage would be in a joint with no defects. This phenomenon has been reported when articular chondrocytes sustain damage, are unable to repair themselves, and de-differentiate into fibrotic chondrocytes.

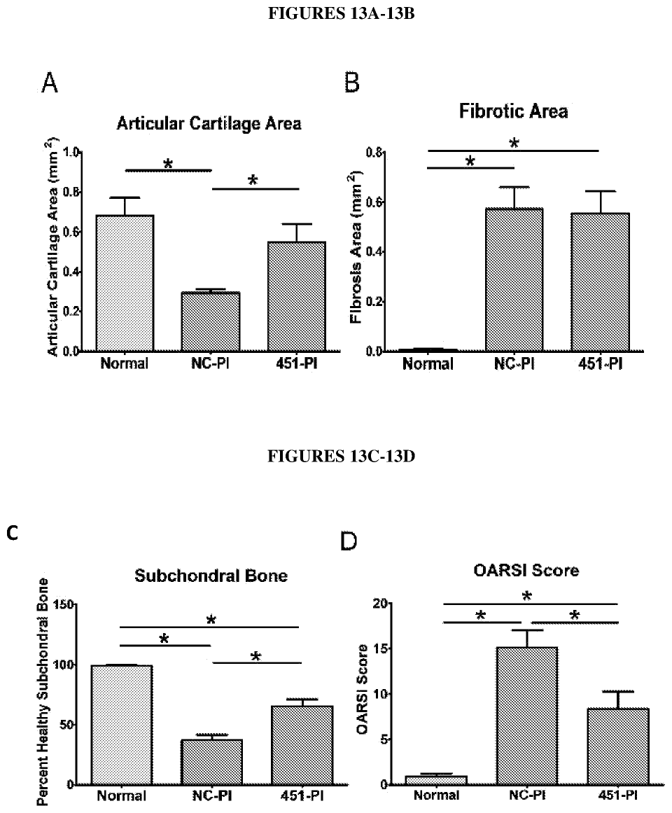

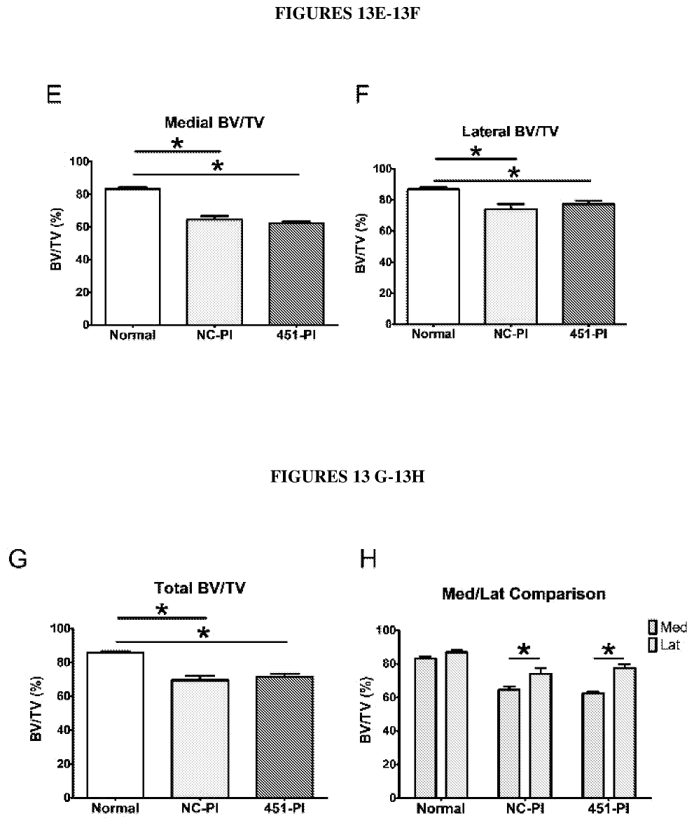

Histomorphometric results are shown in FIGS. 13A-13H. Quantitative histomorphometry showed OA was present in both limbs that underwent bilateral ACLT surgery, regardless of treatment (FIG. 13A-13C). Reduction in articular cartilage area was greatest in the NC-PI limbs (FIG. 13A). The fibrotic area in the ACLT legs was significantly increased compared to normal limbs with no difference between treatments (FIG. 13B). Subchondral bone quality was reduced in ACLT limbs, with effects on the NC-PI legs being greatest (FIG. 13C). Similarly, the OARSI scores in ACLT legs were significantly higher than in normal legs, and the OARSI score for NC-PI limbs was higher than for 451-PI limbs (FIG. 13D). Micro-CT analysis supported these findings. Medial BV/TV was reduced to a similar extent in ACLT limbs, with no differences evident due to treatment (FIG. 13E). This was also the case for lateral BV/TV (FIG. 13F) and for total BV/TV (FIG. 13G). Subchondral bone damage was greater in medial condyles in ACLT limbs (FIG. 13H), though both medial and lateral condyles were affected when compared to controls (FIG. 13E, 13F). Taken together, prophylactic treatment with 451-PI was able to reduce OA severity.

451-PI does not Reverse OA Progression.

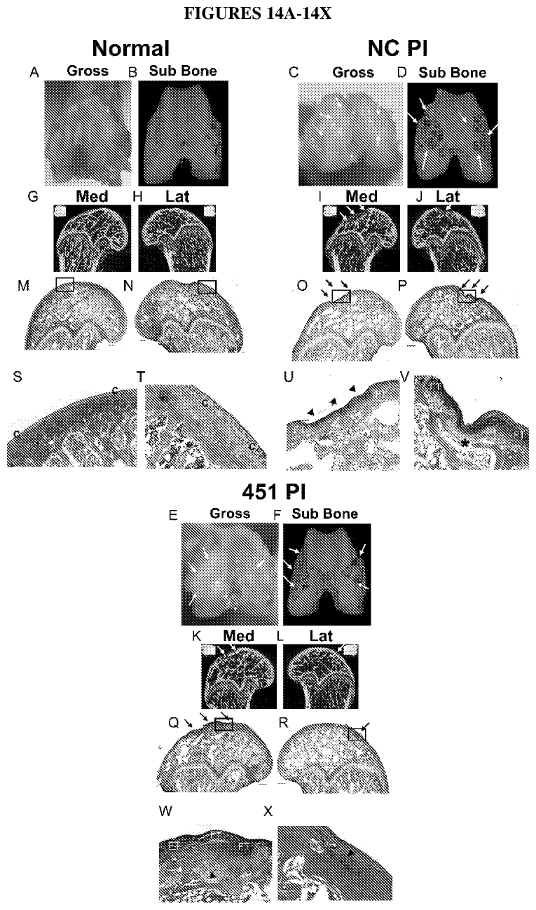

When 451-PI injections were initiated on post-surgery day 24 and continued twice weekly for 3 weeks, there was no evidence that the damage caused by ACLT was reversed, as shown in FIGS. 14A-14X. Gross images and 3D micro-CT reconstructions of femurs of the control (FIG. 14A, 14B), NC-PI (FIG. 14C, 14D), and 451-PI (FIG. 14E, 14F) showed evidence of OA in ACLT limbs, demonstrated by cartilage legions and subchondral bone erosion (white arrows) in both NC-PI and 451-PI limbs. This finding was corroborated in micro-CTs of medial and lateral femurs. The controls had a healthy subchondral bone plate with no evidence of erosion (FIG. 14G, 14H). Conversely, the subchondral bone plate of NC-PI (FIG. 14I, 14J) and 451-PI (FIG. 14K, 14L) limbs showed evidence of erosion and remodeling (white arrows).

Masson's trichrome stained medial and lateral femurs showed normal cartilage morphology in controls (FIG. 14M, 14N), while NC-PI (FIG. 14O, 14P) and 451-PI (FIG. 14Q, 14R) treated limbs exhibited cartilage damage and subchondral bone remodeling (black arrows). At higher magnification, smooth articular surface (C) and healthy subchondral bone plates were observed in controls (FIG. 14S, 14T). Higher magnification of NC-PI (FIG. 14U, 14V) and 451-PI (FIG. 14W, 14X) femurs revealed abnormal cartilage remodeling (black arrowheads), fibrotic tissue formation (FT), and microfractures (asterisks) in the subchondral bone. No obvious differences could be detected qualitatively in OA severity between NC-PI and 451-PI limbs.

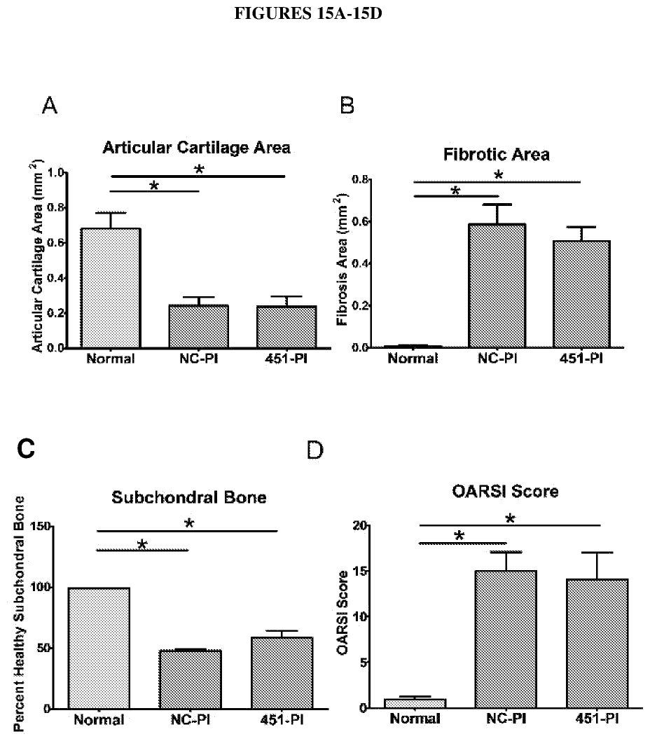

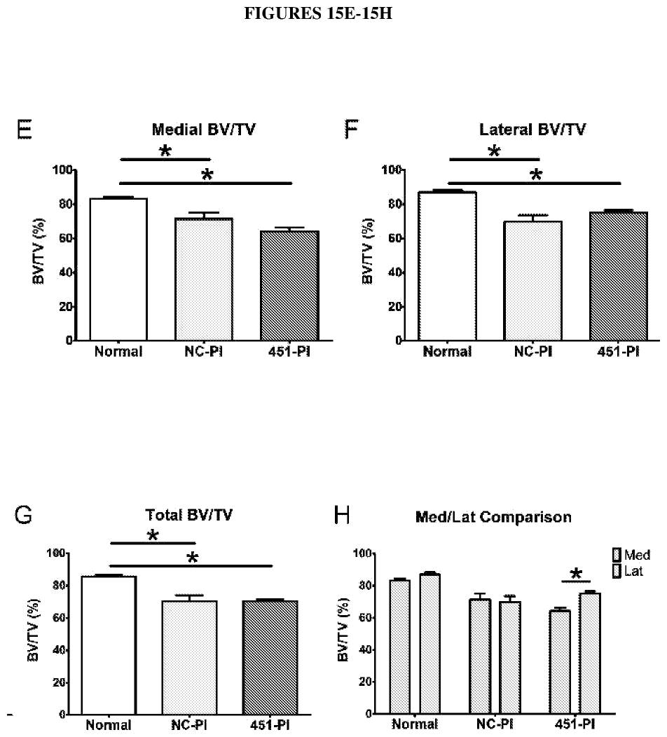

Quantitative histomorphometry confirmed that OA was present following ACLT surgery, regardless of treatment, as shown in FIGS. 15A-15H. Both NC-PI and 451-PI treated limbs had decreased articular cartilage area (FIG. 15A), increased fibrotic area (FIG. 15B), decreased quality of subchondral bone (FIG. 15C), and increased OARSI scores (FIG. 15D) compared to controls. There were no differences between treatment limbs, based on histomorphometry. Medial BV/TV was reduced to a similar extent in the ACLT limbs compared to controls (FIG. 15E). This was also the case for lateral BV/TV (FIG. 15F) and total BV/TV (FIG. 15G). Interestingly, subchondral bone damage was greater in the medial condyle in the 451-PI treated limb, but not the NC-PI treated limb (FIG. 15H). OA did not affect force generation in the TA muscles as measured by maximum twitch and tetany in ACLT limbs compared to normal controls (FIG. 15A-15D). Taken together, therapeutic treatment with 451-PI did not reverse OA progression.

Prophylactic Treatment with miR-122 does not Mitigate OA Progression.

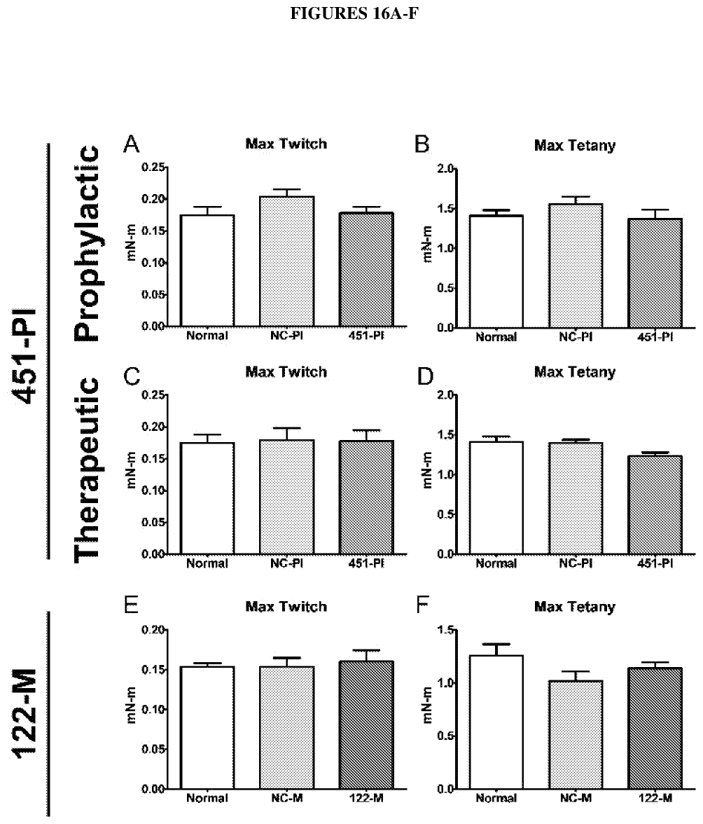

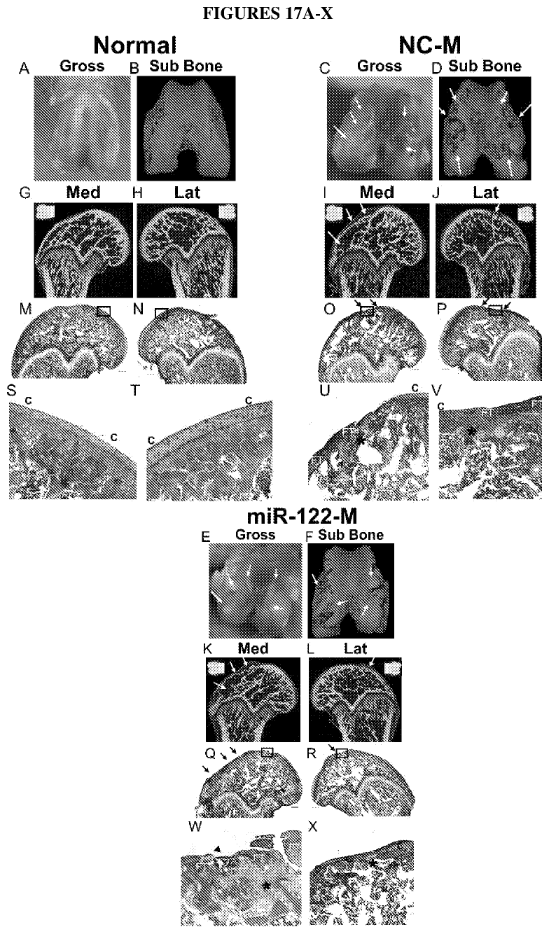

When miR-122 mimic injections were given on post-surgery days 3, 12, and 21, there was no apparent reduction in the severity of OA caused by ACLT detected, as shown in FIGS. 16A-16F. Bone morphology was analyzed further, as shown in FIGS. 17A-17X. Gross images and 3D micro-CT reconstructions of femurs controls showed healthy cartilage and subchondral bone morphology (FIG. 17A, 17B). In contrast, NC-PI (FIG. 17C, 17D) and 122-M treated limbs (FIG. 17E, 17F) exhibited cartilage legions and subchondral bone damage (white arrows). This was confirmed in micro-CTs of the medial and lateral femurs. No evidence of subchondral bone irregularities was seen in the normal aged controls (FIG. 17G, 17H). However, ACLT limbs had evidence of subchondral bone remodeling and erosion (white arrows, FIG. 17I-17L).

Masson's trichrome stained histology of the medial and lateral femurs showed normal cartilage and subchondral bone morphology in controls limbs (FIG. 17M, 17N), whereas there were abnormal cartilage remodeling (black arrowhead), fibrotic tissue formation (FT), and microfractures in the subchondral bone plate (asterisk) in the NC-M (FIG. 17O, 17P), and 122-M (FIG. 17Q, 17R) treated limbs. At higher magnification, healthy cartilage (C) and normal subchondral bone plate were seen in the controls (FIG. 17S, 17T). The higher magnification of NC-M (FIG. 17U, 17V) and 122-M (FIG. 127W, 17X) treated limbs displayed abnormal cartilage remodeling (black arrowhead), fibrotic tissue formation (FT), and microfractures in the subchondral bone plate (asterisk). There was no discernable qualitative difference in OA severity between treatments.

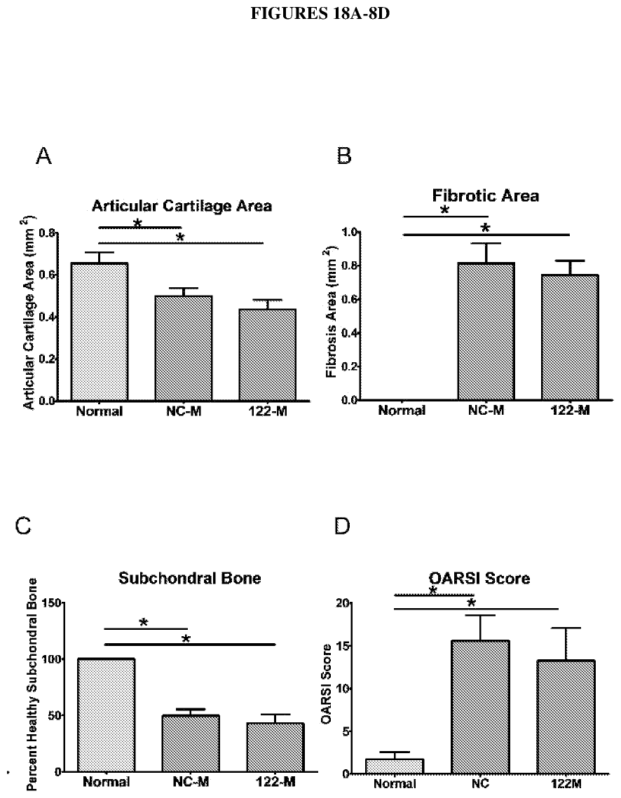

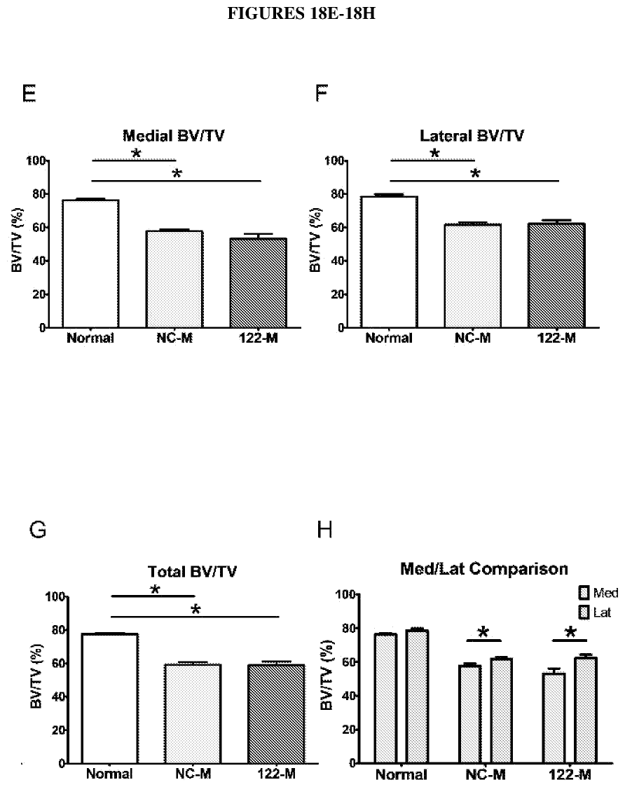

Quantitative histomorphometry showed that OA was present in both limbs that underwent the bilateral ACLT surgery, as shown in FIGS. 18A-18D). Both NC-M and 451-M treated limbs had decreased articular cartilage area (FIG. 18A), increased fibrotic area (FIG. 18B), decreased quality of subchondral bone (FIG. 18C), and increased OARSI scores (FIG. 18D) compared to controls. There were no differences between treatment limbs analyzed in FIGS. 18A-18D. These data were corroborated by the micro-CT analysis, which are shown in FIGS. 18E-18H. Medial BV/TV was reduced to a similar extent in the ACLT limbs compared to controls (FIG. 18E). This was the case for lateral BV/TV (FIG. 18F) and total BV/TV (FIG. 18G). Similar to our earlier findings, subchondral bone damage was greater in the medial condyles in ACLT limbs (FIG. 18H), though both medial and lateral condyles had decreased subchondral bone when compared to the control FIG. 18E-18F). OA did not affect force generation in the TA muscles as measured by maximum twitch and tetany in ACLT limbs compared to normal controls that were shown in FIG. 16E-16F. Taken together, these data suggest that 122-M treatment was not able to mitigate OA disease progression when given with the current dosing regimen.

### Discussion

Increasing evidence indicates that microRNA profiles in disease pathologies differ from their healthy tissue counterparts and play a role in cancer, cardiovascular diseases, and rheumatoid arthritis, as well as osteoarthritis (see Lian et al. Cell Death Dis. 2018; 9(9):919; Li et al, supra; and Mirzamohammadi et al. Curr Osteoporos Rep. 2014; 12(4):410-9). miR-140 attenuates early-stage OA progression by protecting against chondrocyte senescence¹³⁰, while miR-204 and miR-211 ablation accelerate OA progression by increasing RUNX2 and osteogenic differentiation of mesenchymal progenitor cells (see Si et al. Mol Ther Nucleic Acids; 2020; 19(37):15-30; and Huang et al. Nat Commun. 2019; 10(1):1-13).

These observations speak to the numerous pathways affected during OA progression. Our research has shown that miR-451 is specifically increased in OA articular cartilage, although in vitro studies indicate that it does not induce apoptosis or compromise cell viability in chondrocytes (see Scott, supra; and Tang et al. Mol Med Rep. 2018; 18(6):5295-301). However, in the presence of IL-1β, miR-451 exacerbates the production of catabolic MMP-13 and inflammatory PGE2. This suggests that miR-451 cross-talks and synergistically stimulates downstream targets of the IL-1β signaling pathway that are only present after IL-1β stimulation or in OA. The identities of these specific downstream targets remain unclear using a TargetScan analysis (TargetScan Human Prediction of microRNA targets; for further details see the website found at www.targetscan.org/vert_71/).

The elevated presence of both IL-1β and miR-451 in OA makes this a novel target for OA therapies. Our findings indicate that inhibiting miR-451 soon after injury and sustained over six weeks prevents OA progression in a severe bilateral ACLT model in rats. This observation was supported with histomorphometrics of the articular cartilage area, percent healthy subchondral bone and OARSI scoring. While early and sustained intra-articular injections using a specific miR-451 inhibitor were clearly beneficial, no differences were seen in the fibrotic area covering the defect when compared to the contralateral limb treated with a control injection. This indicates that the target for miR-451 does not include pathways that regulate fibrosis.

Fibrotic cartilage generation during cartilage repair in the progression of OA is well documented. Articular cartilage repair has been attempted in the last several decades using processes such as inhibiting or stimulating various cellular pathways and cell-based strategies using mesenchymal stem cells (MSCs) in cartilage lesions. While improved outcomes have been demonstrated with these strategies, the most prominent issue is the development of fibrotic cartilage instead of hyaline cartilage. Fibrocartilage has different mechanical properties and leads to detrimental outcomes in articular cartilage regeneration strategies (see Zhang et al. Am J Transl Res. 2019; 11(10):6275-89). Therefore, it is noteworthy that inhibition of miR-451 did not significantly increase fibrocartilage generation above levels associated with OA.

Histomorphometric analysis of the subchondral bone plate showed that treatment with 451-PI was able to achieve subchondral bone closer to normal controls. This finding was not evident in the micro-CT analysis of the subchondral bone plate, due to the difference in analytical methods. The histomorphometric analysis provides insight into the health and quality of the subchondral bone plate as determined by subchondral bone remodeling, micro-fractures, and osteophyte formation. The micro-CT analysis does not allow us to determine the quality of the bone, only the total amount of bone present. Therefore, it is not surprising that there were differences in these metrics for the prophylactic cohort. The micro-CT analysis indicated a greater difference in the subchondral bone plate in the medial femoral condyles compared to the lateral femoral condyles, as is supported by the literature for this model.

Interestingly, miR-451 inhibition after OA onset (therapeutic cohort) does not appear to reduce OA severity. This may be due to the injection duration (3 total weeks of therapeutic injections as compared to 6 weeks), or to the fact that reversing OA after it has developed has never been achieved to date. A longer duration of 451-PI treatment after OA has developed is contemplated and may improve the outcome.

miR-122 enhances the sensitivity of hepatocellular carcinoma to the chemotherapy drug oxaliplatin by targeting the WNT/β-catenin pathway. The heavy involvement of WNT/β-catenin signaling in OA has been prevalent in the literature and studies have shown the activation of β-catenin signaling results in mouse chondrocytes undergoing phenotypical changes similar to that seen in OA. Knockout of the extracellular WNT-signaling antagonist FrzB caused enhanced expression of MMPs and accumulation of β-catenin in chondrocytes stimulated with IL-1β, which is supported by FrzB null mice having severe cartilage loss compared to wild type control mice in both post-traumatic and enzymatic models of OA.

An analysis of miR-122's targets using the database TargetScan indicated that miR-122 may cross-talk with WNT/β-catenin signaling by targeting FOXO3 (Forkhead O3), FOXP2, and FOXK2. Additionally, miR-122 may be influencing chondrocyte maturation and homeostasis markers such as SOX6 (sry-box transcription factor 6), IHH (Indian hedgehog), ADAM10 (ADAM metallopeptidase domain 10), and HIF3A (hypoxia-inducible factor 3-alpha). miR-122's ability to prevent downstream effects of IL-1β stimulation in vitro makes it an attractive option for treatment for OA.

Various methods of delivery of microRNAs are contemplated. Over the last couple of decades, years of research have gone into therapeutic oligonucleotides, but only a handful have received market approval. These work particularly well in vaccine development, as they need only present the oligonucleotide for a short period to initiate an immune response before they are destroyed. The most recent and well-known examples are the new immerging mRNA-bases vaccines for the ZVIKA, EBOLA, and SARS-CoV2 viruses. These lend value to the beneficial applications of oligonucleotide therapies but highlight the current limitations with local administration, liver accumulation, and rapid destruction.

The introduction of chemical modifications such as locked nucleic acids (LNA) allowed better outcomes for microRNA inhibition delivery; however, the literature is still lacking widespread studies that deliver microRNAs mimics. Our 451-PI had specific proprietary chemical modifications that provided extra stability and allowed our inhibitor to enter the cell unaided through a process known as gymnosis. We used a different dosing regimen for the miR-122 mimic, and while there were modifications to the structure, they were not as robust as those to the inhibitor. Our results did not detect a difference in OA severity with 122-M treatment compared to NC group. It is possible that miR-122 was destroyed before it could elicit its effects, the dosing concentration was too low, or the dosing was too infrequent. Longer dosing regimens and/or increased dose concentration are contemplated. Using a fluorophore to our mimic to track location and persistence after administration in experimental dosing studies is also contemplated.

Muscle wasting due to decreased mobility and eventually compromised joint stability may impact OA progression. Advanced hip and knee OA patients have lower limb muscle weakness and increased muscle atrophy. A 10% reduction in the gastrocnemius area in OA rats who underwent an ACLT surgery has been reported, but no studies have examined how the tibialis anterior muscle is affected in this model. We evaluated the twitch and tetany muscle force in the tibialis anterior in both studies and did not see any differences between any of the groups. The degree of atrophy in the tibialis anterior muscle may have not caused a functional decline, the tibialis anterior muscle may be less susceptible to atrophy, it may not be the primary muscle affected in OA-induced atrophy, or it may take longer than 6 weeks for muscle atrophy to propagate to the tibial portion of the limb in OA progression.

Thus, 451-PI mitigated OA progression compared to NC-PI limbs in the prophylactic cohort based on histomorphometric analysis and OARSI scoring, but no differences were detected by micro-CT. 451-PI treatment beginning 24 days post-surgery did not produce a detectable reduction of OA severity and 122-M injections did not produce a detectable reduction in OA severity compared to NC-M limbs.

### Conclusion

Treatment with 451-PI had preventative effects on OA progression when administered at the time of injury. However, no therapeutic effect was detected when 451-PI was administered after OA had developed. Mitigation of OA progression was not detected with our current dosing regimen with 122-M, thus higher and/or more frequent dosing is contemplated. The Examples of the invention present multiple studies that examine the therapeutic potential of inhibiting the dysregulated inflammatory pathways of IL-1β and TNF-α that control both the chronic inflammatory responses as well as the production of catabolic matrix-degrading enzymes for the treatment of OA. These studies highlight the role that chronic inflammatory signaling play on OA, and how targeting the disease at its root has viable therapeutic potential to prevent OA progression.

A unilateral and bilateral ACLT model of OA was established to evaluate different therapeutics. This was essential for creating and understanding this specific model of OA. Another goal was to evaluate the therapeutic potential of 24R,25 treatment in a severe model of OA. While 24R,25 treatments can be effective in a mild model of OA, treatment with 24R,25 in a severe model did not provide evidence of mitigation of OA progression.

The Examples demonstrate the use of miRs as novel therapeutics for the treatment of OA. miR-122 and miR-451 were selected based on concurrent RNAseq data in our lab. We hypothesized that miR-122 was able to maintain the resting zone chondrocytes and miR-451 was driving terminal differentiation and that this may be a viable target for the aberrant signaling present in OA. We found that miR-122 is able to prevent the inflammatory response to both IL-1β and TNF-α stimulation and prevent the production of downstream targets in primary rat articular chondrocytes. Since protective effects of miR-122 treatment on OA were not detectable in the animal model of bilateral OA, other modes of delivery are contemplated. Molecules such as miR-122 or miR-122 mimic may be packaged for delivery using various technologies that are known in the art. For example, lipid nanoparticles may be used to encapsulate the miR molecules, such as the packaging used for mRNA vaccines. The most recent and well-known examples are the delivery systems for new immerging mRNA-bases vaccines for the ZVIKA, EBOLA, and SARS-CoV2 viruses. Another option is the use of chemically modified antisense oligonucleotides (ASOs), which are short synthetic nucleic acids that hybridize with cellular RNA using classic Watson-Crick base pairing to modulate gene expression. Artificial miRNA-enabled gene regulation relies on sequence complementarity between the target mRNA 3′ untranslated region and the first seven to eight 5′-nucleotides of the miRNA (the seed sequence). mRNA-miRNA binding outside the miRNA seed is variable, such that a single miRNA may interact with multiple mRNAs with different affinities. ASO-RNA binding is rigorously regulated by complementarity between target RNA and the full ASO molecule, which is typically between 13 and 30 nucleotides in length. This stringent binding specificity directly correlates with ASO efficacy. The miRs of the invention may also be incorporated into a viral vector, such as an adeno-associated vector (AAV), a lentivirus, an adenovirus, or any other gene therapy viral vector.

The various modes of delivery of miRs lend value to the beneficial applications of oligonucleotide therapies but highlight the current limitations with local administration, liver accumulation, and rapid destruction. Without being bound by theory, the miR-122 may be destroyed before it elicits its effects, the dosing concentration may need to be increased, or the dosing may need to be administered more frequently. Additionally, while the Examples suggest that miR-122 is acting through a means that can target both IL-1β and TNF-α signaling pathways, the precise mechanism of action still remains to be elucidated.

The results with miR-451 were the most surprising. This microRNA was originally chosen as a negative control. Interestingly, miR-451 transfection exacerbated the IL-1β stimulated increase in cytokines, chemokines, and catabolic enzymes even more so than IL-1β stimulation alone. However, miR-451 transfection had no detectable effect on these same molecules following TNF-α stimulation using the methods of analysis described herein. These data indicate that miR-451 is targeting an anti-inflammatory molecule that interferes with the IL-1β signaling pathway, but not the TNF-α signaling pathway. Additionally, miR-451 expression was elevated in the cartilage of OA in Sprague Dawley rats, indicating that inhibiting miR-451 is a feasible target for alleviating OA. Prophylactic administration of miR-451 inhibitor in the severe bilateral model of OA in Sprague Dawley rats produced a significant decrease in OA severity compared to vehicle injections. These results were after only twice-weekly injections for a duration of 6 weeks. This frequency was determined based on a previously published protocol for a different microRNA. In some embodiments of the invention the dosing regimen (dosing concentration or frequency) can be altered to achieve an even more robust response or decreased to make miR-451 administration cost-optimal and clinically scalable. A dosing experiment where miR-451 is tagged with a fluorophore to determine the duration miR-451 persists in the joint space is contemplated.

In summary, the invention is a method for administering or inhibiting microRNAs to regulate the dysregulated processes responsible for OA disease progression. While mitigation of OA progression in vivo in response to administration of miR-122 mimic, the robust in vitro data indicate there is great potential for this microRNA as a therapeutic. Prophylactic administration of the miR-451 inhibitor resulted in a decreased OA severity in the severe bilateral OA model. While the invention has been described in terms of its preferred embodiments, those skilled in the art will recognize that the invention can be practiced with modification within the spirit and scope of the appended claims. Accordingly, the present invention should not be limited to the embodiments as described above but should further include all modifications and equivalents thereof within the spirit and scope of the description provided herein.

We claim:

1. A pharmaceutical composition for use as an inhibitor of osteoarthritis, comprising at least one agent selected from the group consisting of a microRNA-122 (miR-122), a miR-122 mimic and an inhibitor of a microRNA-451 (miR-451), wherein an inflammatory response is inhibited in articular chondrocytes.

2. The pharmaceutical composition of claim 1, wherein delivery of the pharmaceutical composition to an injured joint inhibits development or progression of the osteoarthritis.

7. The pharmaceutical composition of claim 2, wherein the joint has received an acute injury.

3. The pharmaceutical composition of claim 2, wherein the pharmaceutical composition is administered by injection into the joint.

4. The pharmaceutical composition of claim 1, wherein delivery of the pharmaceutical composition is sustained for at least six weeks.

5. The pharmaceutical composition of claim 1, wherein the inhibitor of microRNA-451 modulates miR-451-mediated inflammatory processes of osteoarthritis disease progression.

6. The pharmaceutical composition of claim 1, wherein the miR-122 and/or miR-122 mimic inhibits an inflammatory response to IL-113 and TNF-α stimulation in the articular chondrocytes.

7. The pharmaceutical composition of claim 6, wherein the at least one agent is packaged with antisense-oligonucleotides and/or lipid nanoparticles for delivery to at least one joint.

8. A method of treating or inhibiting osteoarthritis in a subject in need thereof, comprising the step of administering a therapeutically sufficient amount of a pharmaceutical composition comprising at least one agent selected from the group consisting of a microRNA-122 (miR-122), a miR-122 mimic and an inhibitor of microRNA-451 (miR-451).

9. The method of claim 8, wherein the therapeutically sufficient amount of the pharmaceutical composition is able to inhibit an inflammatory response in articular chondrocytes.

10. The method of claim 9, wherein the subject has sustained an injury to at least one joint.

11. The method of claim 9, wherein the step of administering the pharmaceutical composition is sustained for at least six weeks.

12. The method of claim 9, wherein the at least one agent is packaged with antisense-oligonucleotides and/or lipid nanoparticles for delivery to at least one joint.
